(19) United States
(12) Patent Application Publication
(10) Pub. No.: US 2026/0083712 A1
(43) Pub. Date: Mar. 26, 2026

(54) METHODS FOR TREATING PATIENTS WITH OCULAR COMORBIDITIES OF AN AUTOIMMUNE DISORDER

(71) Applicant: Iolyx Therapeutics, Inc., Burlingame, CA (US)

(72) Inventors: Erin NEWMAN, Burlingame, CA (US); Elizabeth W. JEFFORDS, Hillsborough, CA (US); Houman David HEMMATI, Santa Monica, CA (US); Heather BERGER, Burlingame, CA (US); Shannon DAHL, Palo Alto, CA (US)

(73) Assignee: Iolyx Therapeutics, Inc., Burlingame, CA (US)

(21) Appl. No.: 19/334,559

(22) Filed: Sep. 19, 2025

Related U.S. Application Data
(60) Provisional application No. 63/697,723, filed on Sep. 23, 2024.
(60) Provisional application No. 63/764,664, filed on Feb. 28, 2025.
(60) Provisional application No. 63/773,858, filed on Mar. 18, 2025.
(60) Provisional application No. 63/808,253, filed on May 19, 2025.

(51) Int. Cl. A61K 31/44; A61K 9/00; A61P 27/02

(52) U.S. Cl. CPC A61K 31/44; A61K 9/0048; A61P 27/02

(57) ABSTRACT

Methods of administering ophthalmic pharmaceutical formulations of the phosphodiesterase-4 inhibitor, roflumilast, for treating patients with ocular comorbidities of autoimmune disorder, including dry eye disease. The methods include selecting a patient previously diagnosed with an autoimmune disorder and administering an ophthalmic pharmaceutical composition comprising roflumilast to the patient to treat the ocular comorbidity of the autoimmune disorder. The method can result in a reduction of a total lissamine green conjunctival staining score or a reduction in total corneal fluorescein staining score relative to baseline.

### CROSS REFERENCE TO RELATED APPLICATIONS

The present application claims priority to U.S. Provisional Application No. 63/808,253, filed May 19, 2025, U.S. Provisional Application No. 63/773,858, filed Mar. 18, 2025, U.S. Provisional Application No. 63/764,664, filed Feb. 28, 2025, and U.S. Provisional Application No. 63/697,723, filed Sep. 23, 2024. Each of these applications is incorporated by reference herein in their entirety.

### FIELD OF THE INVENTION

The present invention relates to methods of treatment using stable and pharmaceutically effective ophthalmic compositions of the phosphodiesterase-4 inhibitor, roflumilast. More specifically, the invention relates to methods of treating ocular comorbidities of an autoimmune disorder, including dry-eye disease and ocular pain, by administering ophthalmic pharmaceutical compositions of roflumilast to a patient diagnosed with the autoimmune disorder.

### BACKGROUND OF THE INVENTION

Roflumilast is a potent and selective long-acting inhibitor of phosphodiesterase (PDE) type 4, with anti-inflammatory and potential antineoplastic activities. Roflumilast is known to be suitable as a bronchial therapeutic agent as well as for the treatment of inflammatory disorders. Compositions containing roflumilast are used in human and veterinary medicine and have been proposed for the treatment and prophylaxis of diseases including but not limited to: inflammatory and allergen-induced airway disorders (e.g., bronchitis, asthma, COPD), dermatoses (e.g., proliferative, inflammatory, and allergen induced skin disorders), and generalized inflammations in the gastrointestinal region (Crohn's disease and ulcerative colitis). Oral pharmaceutical compositions of roflumilast are currently marketed under the tradenames Daliresp® (in the United States) and Daxas® (in Europe) for COPD, and topical compositions of roflumilast cream for dermatological use are currently marketed under the tradename Zoryve™ (in the United States) for psoriasis, atopic dermatitis, and seborrheic dermatitis.

Roflumilast and its synthesis are described in U.S. Pat. No. 5,712,298. While the therapeutic effectiveness of oral and dermal pharmaceutical compositions have been studied, there is a need for ophthalmic pharmaceutical compositions of roflumilast suitable for treating ocular disorders.

In general, the delivery of drugs to the eye is very difficult, as pharmaceutical ophthalmic agents must balance tolerability, sterility, safety, and efficacy. See, e.g., Priyanka Agarwal et al., Formulation Considerations for the Management of Dry Eye Disease, Pharmaceutics, 13, 207 (Feb. 3, 2021). Patients having autoimmune disorders are known to suffer from a variety of ocular comorbidities. See Glover K. et al., Epidemiology of Ocular Manifestations in Autoimmune Disease, Front Immunol. (2021). One exemplary ocular comorbidity of autoimmune disorders discussed herein is dry eye disease. Nearly half of patients with dry eye disease, uveitis, and scleritis have an underlying autoimmune disease, and an underlying autoimmune disease such as Sjogren's Disease has a further association with potentially vision-threatening side effects such as corneal melt, corneal vascularization, uveitis, retinal vasculitis, and optic neuritis. See Henrich 2014, Akpek 2009 and Xiong 2024. Conversely, dry eye patients also have a much higher risk of also having one or more autoimmune diseases than non-dry eye patients, including rheumatoid arthritis (1.94 OR), systemic lupus erythematosus (SLE) (4.21 OR), Crohn's disease (2.01 OR), Thyroid or Graves' disease (1.73 and 4.58 OR respectively), Sjögren's disease (60.3OR), and others. (Vehof 2021). The dry eye immune response includes interplay between the conjunctiva and lymph nodes, which are directly connected via lymphatic vessels. See Stern & Pflugfelder 2017.

The literature reports that patients having autoimmune disorders such as Sjogren's disease frequently have more severe ocular comorbidities (Cui 2021, Xiong 2024, Henrich 2014, McCoy 2022), and are less likely to achieve treatment endpoints such as resolution of staining than those without Sjogren's (Cui, 2021), and an association with vision threatening ocular complications such as vascularization, scarring, and ulceration. (Xiong, 2024). For example, Henrich 2014 reports that patients with systemic autoimmune disease are classified with having more severe dry eye disease than those without systemic autoimmune disease (88.9% vs 74.2%). Patients with thyroid dysfunction also demonstrate more severe dry eye manifestations, including higher OSDI scores, higher tear ferning grades, and shorter tear breakup times compared with healthy controls (Alanazi 2019). In some cases, thyroid eye disease contributes to lower tear volume, higher conjunctival staining, and more frequent orbital inflammation (Henrich 2014; Cui 2021). These findings highlight that thyroid-related autoimmune disease, like Sjögren's, represents a subgroup with higher severity, poorer treatment responsiveness, and increased risk of progressive ocular morbidity. Treating ocular comorbidities of autoimmune disease presents additional challenges due to the increased severity and reduced responsiveness of their eye conditions due to the existing comorbidities. Ophthalmic treatment options often have negative side effects that may exacerbate systemic autoimmune symptoms and quality of life, or interact negatively with other therapies, complicating overall management and treatment of these disorders. Patients with autoimmune disorders are also often less frequently included in clinical trials, as their baseline disease severity and use of concomitant systemic immunomodulators often lead to exclusion, limiting the evidence base for this population. Hence, it is often difficult to balance and optimize systemic treatments which can positively impact the systemic autoimmune disease concurrently with ocular treatments which can positively impact the ocular comorbidities. As such, identifying an autoimmune etiology for dry eye patients can play an important part of understanding the disease, and in the differentiation of targeted treatment choice.

Moreover, certain autoimmune disorders, such as Sjogren's disease, multiple sclerosis, coeliac disease, Crohn's disease, Grave's disease and other thyroid dysfunctions, rheumatoid arthritis, and sarcoidosis, disproportionately affect female patients. See Ngo 2014, Varma 2016, Sen 2015, Steren 2022, Akpek 2009, Rosenbaum 2019. Additionally, females generally experience more ocular disorders and vision threatening disease (Aninye 2021). Thus, there is a heightened need to treat ocular comorbidities of autoimmune disorders, specifically in female patients. Existing therapies are either inadequate or are not targeted to treat this patient population.

### SUMMARY OF THE INVENTION

The present invention relates to the administration of ophthalmic pharmaceutical compositions of the phosphodiesterase-4 inhibitor, roflumilast, for use in treating patients with ocular comorbidities of autoimmune disease. The inventors of the subject application have determined that targeting treatment of ocular comorbidities, including dry eye, ocular pain, and thyroid eye disease, in patients with autoimmune disease, can allow for continuous use of existing, systemic medications for the treatment of the underlying autoimmune disease while providing direct ophthalmic treatment of the comorbidities occurring in the eye. The methods disclosed herein can offer an improvement to using either existing systemic autoimmune treatments, or less optimal ophthalmic treatments to treat the ocular comorbidities of autoimmune disease, both of which are often insufficient in treating moderate and severe dry eye disease, among other ocular comorbidities.

In existing treatment methods, patients suffering from autoimmune disorders can be on systemic medication, and notably even when they experience some level of control of their systemic disease, they continue to present with troublesome and clinically significant ophthalmic comorbidities. That is, the existing treatments involving systemic medication are not adequate for treatment of the ocular comorbidity. Moreover, patients suffering from autoimmune disorders are often excluded from clinical trials due to their use of systemic medication. In contrast, the methods disclosed herein comprise the administration of roflumilast locally to the patient to address the ocular comorbidity which would otherwise remain sub-optimally controlled.

Addressing ocular complications locally, using the compositions described herein, without the need for additional systemic medication or non-specific ophthalmic treatments, can avoid myriad issues, including complications due to existing autoimmune treatments, compounded side effects, drug-drug interactions, and contra-indications. Additionally, the disclosed compositions and methods may also be used in an additive or adjuvant manner alongside systemic therapies for the underlying autoimmune disease, enabling patients to maintain necessary systemic treatment without interruption or compromise. The administration of roflumilast locally to the patient is believed to result in low plasma levels as reflected in the preclinical studies discussed herein. As a result, the methods disclosed herein can provide an improvement to existing therapies while avoiding undesirable complications or side effects.

In certain embodiments, a method is provided for locally treating an ocular comorbidity of an autoimmune disease in a patient having an autoimmune disease. The method comprises: (a) selecting a patient previously diagnosed with the autoimmune disease; and (b) administering an ophthalmic pharmaceutical composition comprising roflumilast to the patient to treat the ocular comorbidity of the autoimmune disease.

In certain embodiments, the ocular comorbidity of autoimmunity is selected from the group consisting of diseases of both the ocular surface/anterior compartment as well as the posterior compartment of the eye, including primary or secondary dry eye disease, keratitis, corneal ulcers, conjunctivitis, blepharitis, scleritis, episcleritis, uveitis (pan, posterior, anterior, intermediate, or uveitis associated with a systemic disease such as Juvenile Idiopathic Arthritis related uveitis), neurotrophic keratitis, corneal wound healing, corneal melt, glaucoma, cataracts, retinal vasculitis, optic neuritis, macular edema, retinal vein occlusion, choroiditis, retinal detachment, vitreous/retinal hemorrhaging, optic atrophy, photophobia, diplopia, thyroid eye disease, diabetic retinopathy, diabetic macular edema, geographic atrophy, or ocular pain (including for example, idiopathic, neuropathic, nociceptive, nociplastic, neuropathic-nociceptive, inflammatory, mixed-mechanism, psychogenic, or multifactorial in origin). In preferred embodiments, the ocular comorbidity is primary dry eye disease or dry eye disease secondary to an additional ophthalmic condition, neurotrophic keratitis, uveitis, diabetic retinopathy, ocular pain, or thyroid eye disease. In especially preferred embodiments, the ocular comorbidity of autoimmune disease is primary or secondary dry eye disease.

In certain embodiments, a method is provided for treating dry eye disease in a patient. The method comprises selecting a patient previously diagnosed with an autoimmune disease and having dry eye disease. The method further comprises administering an ophthalmic pharmaceutical composition comprising roflumilast to the patient to treat the dry eye disease.

In certain embodiments, the autoimmune disease is selected from the group consisting of Graves' disease, Hashimoto's thyroiditis, hypothyroidism, hyperthyroidism, or other thyroid dysfunction, mixed connective tissue disease, scleroderma, CREST syndrome (also known as limited cutaneous systemic sclerosis), rheumatoid arthritis, polyarthritis, sero-negative arthritis, anti-cardiolipin antibody positivity, rheumatoid pleurisy, polymyalgia rheumatica, Sjögren's disease, spondylarthrites, ankylosing spondylitis, multiple sclerosis, lupus (systemic lupus erythematosus (SLE) or lupus nephritis (LN)), cutaneous lupus erythematosus, alopecia areata, vitiligo, atopic dermatitis, eczema, psoriasis, psoriatic arthritis, lichen sclerosis, lichen planus, ulcerative colitis, Crohn's disease, diverticulitis, primary biliary cholangitis, type 1 diabetes, latent autoimmune diabetes in adults (LADA), coeliac disease, sarcoidosis, chronic osteoarthritis, primary biliary cholangitis, rosacea, or uveitis (including non-infectious HLA B27 uveitis). In certain embodiments, the patient has one or more autoimmune disorders, a condition known as poly-autoimmunity.

In certain embodiments, an ophthalmic pharmaceutical composition is administered to the patient one or more times daily. In preferred embodiments, the ophthalmic pharmaceutical composition is administered to the patient twice daily.

In certain embodiments, the method results in a reduction of total lissamine green conjunctival staining score in the patient relative to baseline following treatment. In certain embodiments, the reduction is of at least 1 or 2 in total lissamine green conjunctival staining score relative to baseline following treatment.

In certain embodiments, the method results in a reduction of total lissamine green conjunctival staining score in the patient following treatment compared to the same treatment method using a non-active vehicle or placebo. In certain embodiments, the reduction is of at least 1 or 2 in total lissamine green conjunctival staining score compared to the same treatment method using vehicle or placebo.

In certain embodiments, the method results in a reduction of total corneal fluorescein staining score in the patient following treatment compared to the same treatment method using vehicle. In certain embodiments, the reduction is of at least 1 or 2 in total corneal fluorescein staining score compared to the same treatment method using vehicle or placebo

In certain embodiments, the method results in a reduction of total corneal fluorescein staining score in the patient relative to baseline following treatment. In certain embodiments, the reduction is of at least 1 or 2 in total corneal fluorescein staining score relative to baseline following treatment.

In certain embodiments, the method results in a reduction of one or more of the following relative to baseline or compared to a similar method using vehicle: (a) ocular dryness Visual Analog Score (VAS) score; (b) ocular discomfort VAS score; (c) Ocular Surface Disease Index (OSDI)—dry eye (DE) quality of life outcomes; and additional exploratory endpoints including (d) ocular itch VAS score; (e) ocular pain VAS score, (f) hyperemia score; (g) regional corneal fluorescein staining; (h) Schirmer's; or (i) tear-film break up time.

In certain embodiments, the method results in a reduction of symptomatic ocular disease relative to baseline following treatment, including ocular dryness, ocular discomfort, ocular pain, photophobia, ocular itching, or feeling of grittiness in the eye. In certain embodiments, the reduction is of 5 or 10 points on a 100-point VAS score relative to baseline following treatment in ocular discomfort or ocular dryness. In certain embodiments, the methods result in an improvement of Ocular Surface Disease Index (OSDI) score of quality of life following administration relative to baseline. In certain embodiments, the improvement of OSDI score is between 0 and 100 relative to baseline.

In certain embodiments, the method results in a reduction in ocular pain. In certain embodiments, ocular pain is chronic ocular surface pain (COSP). In other embodiments the ocular surface pain is acute. In certain embodiments, the ocular pain can be neuropathic corneal pain. In certain embodiments the ocular pain can be neuropathic nociceptive, or nociplastic pain (see Galor A et al., The Ocular Surface 26:148-56 (2022)). In certain embodiments, the ocular pain can be inflammatory and/or result from a systemic inflammatory condition. In certain embodiments, the ocular pain is associated with eye surgery, including laser-assisted in situ keratomileusis (LASIK) surgery, photorefractive keratectomy (PRK) surgery, cataract surgery, glaucoma surgery, or other surgical procedures, extended contact lens wear, shingles, diabetes, trigeminal neuralgia, radiation therapy, autoimmune or inflammatory disease such as Sjogren's syndrome, ocular graft-versus host disease, or celiac disease. In certain embodiments, the ocular pain is associated with dry eye disease, thyroid disease, or thyroid eye disease. In certain embodiments, features of the ocular pain are manifested as light sensitivity (photophobia), glare or difficulty seeing at night, foreign body sensation, dryness, itch, dysesthesia, burning, prickling, or a crawling feeling on the eye's surface that result in a painful or uncomfortable sensation. In certain embodiments, the degree of pain can be determined by techniques such as the Visual Analog Scale (VAS) ocular pain test, the Ocular Surface Disease Index (OSDI), the Ocular Pain Assessment Survey (OPAS) or the Standardized Patient Evaluation of Eye Dryness (SPEED) technique (see Mehra D et al., Ophthalmol. Ther. 9:427-47 (2020) and Galor supra).

In certain embodiments, the patient maintains a stable or an improved outcome in ocular and non-ocular key safety monitoring assessments relative to baseline, including intraocular pressure assessed by tonometry, corrected visual acuity measured by visual assessment with Snellen chart, external eye examination via slit-lamp biomicroscopy, or assessment of the retina via dilated ophthalmoscopy. In certain embodiments, the ocular tolerability over time with the treatment method is either stable or diminished as treatment progresses.

In certain embodiments, a method is provided for treating ocular pain in a patient. The method comprises administering an ophthalmic pharmaceutical composition comprising roflumilast to the patient to treat the ocular pain. In certain embodiments, the administration results in a reduction of at least 8, 10, 12, 15, 20, 25, or 30 VAS score relative to baseline. In certain embodiments, the ocular pain is chronic ocular surface pain (COSP), acute ocular surface pain, neuropathic corneal pain, neuropathic nociceptive pain, or nociplastic pain. In certain embodiments, the patient has an autoimmune or inflammatory disease. In certain embodiments, the patient has a baseline VAS score of at least 30.

In certain embodiments, an ophthalmic pharmaceutical composition is administered to the patient one or more times daily. In preferred embodiments, the ophthalmic pharmaceutical composition is administered to the patient twice daily. In certain embodiments, the ophthalmic pharmaceutical composition is administered to the patient for at least eight weeks.

### BRIEF DESCRIPTION OF THE DRAWINGS

The accompanying drawings, which are incorporated herein and form part of the disclosure, help illustrate various embodiments of the present invention and, together with the description, further serve to describe the invention to enable a person skilled in the pertinent art to make and use the embodiments disclosed herein. The error bars in the drawings are standard deviation values.

FIG. 1 is a graph depicting the concentration of roflumilast in the conjunctiva after five days administration (four days twice a day (BID), fifth day-single dose (QD)) of the ophthalmic composition of Example 3 (0.1% w/v roflumilast) in Healthy Dutch Belted Rabbits. The x-axis is time in hours, and the y-axis is concentration (ng/g) of roflumilast.

FIG. 2 is a graph depicting the concentration of roflumilast in the cornea after five days administration (four days twice a day (BID), fifth day-single dose (QD)) of the ophthalmic composition of Example 3 (0.1% w/v roflumilast) in Healthy Dutch Belted Rabbits. The x-axis is time in hours, and the y-axis is concentration (ng/g) of roflumilast.

FIG. 3 is a graph depicting the concentration of roflumilast in the plasma after five days administration (four days twice a day (BID), fifth day-single dose (QD)) of the ophthalmic composition in Example 3 (0.1% w/v roflumilast) in Healthy Dutch Belted Rabbits. The x-axis is time in hours, and the y-axis is concentration (ng/ml) of roflumilast.

FIG. 4 provides data from two studies comparing the concentration of roflumilast (ng/g) in the iris ciliary body as a function of time following: (a) a 1% w/v roflumilast formulation administered three times a day (TID); (b) a 0.3% w/v roflumilast formulation administered three times a day (TID); or (c) a 0.1% w/v roflumilast formulation administered two times a day (BID) in Healthy Dutch Belted Rabbits. The x-axis is time in hours, and the y-axis is concentration (ng/g) of roflumilast.

FIG. 5 provides data from two studies comparing the concentration of roflumilast (ng/mL) in the aqueous humor as a function of time following: (a) a 1% w/v roflumilast formulation administered three times a day (TID); (b) a 0.3% w/v roflumilast formulation administered three times a day (TID); or (c) a 0.1% w/v roflumilast formulation administered two times a day (BID) in Healthy Dutch Belted Rabbits. The x-axis is time in hours, and the y-axis is concentration (ng/g) of roflumilast.

FIG. 6 provides data from two studies comparing the concentration of topical roflumilast (ng/g) in the retina/choroid/retina pigment epithelium (RPE) as a function of time following: (a) a 1% w/v roflumilast formulation administered three times a day (TID); (b) a 0.3% w/v roflumilast formulation administered three times a day (TID); or (c) a 0.1% w/v roflumilast formulation administered two times a day (BID) in Healthy Dutch Belted Rabbits. The x-axis is time in hours, and the y-axis is concentration (ng/g) of roflumilast.

FIG. 7 is a graph depicting the total lissamine green conjunctival staining (tLGCS) score and total corneal fluorescein staining (tCFS) score in the right (OD) and left (OS) eyes of a human patient over time following BID administration of the ophthalmic composition of Example 3 (0.1% w/v roflumilast).

FIG. 8 is a graph depicting the total lissamine green conjunctival staining (tLGCS) score and total corneal fluorescein staining (tCFS) score in the right (OD) and left (OS) eyes of a human patient over time following BID administration of the ophthalmic composition of Example 3 (0.3% w/v roflumilast).

FIG. 9 is a graph depicting the mean intraocular pressure (IOP) in a cohort of eleven human patients (at baseline, decreased to n=7 at day 57 of treatment) over time following BID administration of the ophthalmic composition of Example 3 (0.1% w/v roflumilast). The x-axis is mmHg and the Y-axis is the visit number (study day).

FIG. 10 is a graph depicting the total lissamine green conjunctival staining (tLGCS) score and total corneal fluorescein staining (tCFS) score in a cohort of patients (n=11 at baseline, decreasing to n=7 at treatment day 57) over time following BID administration of the ophthalmic composition of Example 3 (0.1% w/v roflumilast).

FIG. 11 is an image of a lissamine green conjunctival staining (LGCS) in a patient at baseline.

FIG. 12 is an image of a lissamine green conjunctival staining (LGCS) in the same patient of FIG. 11 following 57 days of BID administration of the ophthalmic composition of Example 3 (0.1% w/v roflumilast).

FIG. 13 is a graph depicting the mean ocular dryness VAS score, mean ocular discomfort VAS score, and mean Ocular Surface Disease Index (OSDI)—dry eye (DE) symptom outcomes in a cohort patients (n=11 at baseline, decreasing to n=7 at treatment day 57) over the course of a clinical trial involving BID administration of the ophthalmic composition of Example 3 (0.1% w/v roflumilast).

FIG. 14 is a graph depicting the clinical trial design as discussed in Example 10 designed to test key ocular endpoints in a trial of dry eye patients with underlying autoimmune disease.

FIG. 15 is graph depicting the mean total corneal fluorescein staining (tCFS) score change from baseline following administration of an ophthalmic roflumilast composition compared to vehicle at day 15 and day 57 (week 8), as discussed in Example 10.

FIG. 16 is a graph depicting the mean total fluorescein staining (tCFS) scores from baseline following administration of an ophthalmic roflumilast composition compared to vehicle over time through day 57 (week 8), as discussed in Example 10.

FIG. 17 is graph depicting the least squares (LS) mean central corneal fluorescein staining (cCFS) score change from baseline following administration of an ophthalmic roflumilast composition compared to vehicle at day 15 and day 57 (week 8), as discussed in Example 10.

FIG. 18 is a graph depicting the least squares (LS) mean central corneal fluorescein staining (cCFS) scores change from baseline following administration of an ophthalmic roflumilast composition compared to vehicle over time through day 57 (week 8), as discussed in Example 10.

FIG. 19 is a graph depicting the least squares (LS) mean total Lissamine green conjunctival staining (tLGCS) score change from baseline following administration of an ophthalmic roflumilast composition compared to vehicle at day 15 and day 57 (week 8), as discussed in Example 10.

FIG. 20 is a graph depicting the least squares (LS) mean total Lissamine green conjunctival staining (tLGCS) scores change from baseline following administration of an ophthalmic roflumilast composition compared to vehicle over time through day 57 (week 8), as discussed in Example 10.

FIG. 21 is a graph depicting the ocular tolerability at instillation (“instill”) from screening to baseline (BL) at day 1 through day 57. Subjects were administered an ophthalmic roflumilast composition or vehicle, as discussed in Example 10.

FIG. 22 is a graph depicting the intraocular pressure (IOP) readings in subjects administered an ophthalmic roflumilast composition compared to those administered with vehicle from screening to baseline (BL) at day 1 through day 57.

FIG. 23 is a graph depicting the unadjusted change from baseline (CFB) at Day 57 in VAS ocular pain among: all participants (“All Pts”); those participants with baseline VAS ocular pain greater than or equal to 30 at baseline (“VAS Pain≥@BL”); and those with Sjogren's or thyroid disease (SJD/THY).

FIG. 24 is a graph depicting an ANCOVA analysis, using baseline as a covariate, reporting least squared (LS) means change from baseline (CFB) data at Day 57 in VAS ocular pain among all participants (“All Pts”) and those participants with baseline VAS ocular pain greater than or equal to 30 at baseline (“VAS Pain≥30@BL”) for the overall population.

### DETAILED DESCRIPTION OF THE INVENTION

It is to be understood that the invention is not limited to the particular methodology, protocols, and reagents described herein as these may vary. It is also to be understood that the terminology used herein is for the purpose of describing particular embodiments only and is not intended to limit the scope of the present invention which will be limited only by the appended claims. Unless defined otherwise, all technical and scientific terms used herein have the same meaning as commonly understood by one of ordinary skill in the art to which this invention belongs.

All publications, patents and patent applications cited herein are hereby incorporated by reference in their entirety unless otherwise stated. Where the same term is defined in a publication, patent, or patent application and the present disclosure incorporated herein by reference, the definition in the present disclosure represents a controlling definition. For publications, patents and patent applications referenced to describe a particular type of compound, chemistry, etc., the portion relating to such compounds, chemistry, etc. is the portion of the literature incorporated herein by reference.

Note that as used herein, the singular forms “a,” “an,” and “the” include plural referents unless the context clearly dictates otherwise. Thus, for example, “active ingredient” includes a single ingredient and two or more different ingredients.

The term “about” when used in connection with a numerical value is meant to encompass numerical values within a range having a lower limit that is 5% smaller than the indicated numerical value and having an upper limit that is 5% larger than the indicated numerical value.

The term “effective” refers to an amount of a compound, agent, substance, formulation or composition that is of sufficient quantity to result in a decrease in severity of disease symptoms, an increase in frequency and duration of disease symptom-free periods, or a prevention of impairment or disability due to the disease affliction. The amount may be as a single dose or according to a multiple dose regimen, alone or in combination with other compounds, agents or substances. One of ordinary skill in the art would be able to determine such amounts based on such factors as a subject's size, the severity of a subject's symptoms, and the particular composition or route of administration selected.

“Pharmaceutically acceptable” means generally safe for administration to humans or animals. Preferably, a pharmaceutically acceptable component is one that has been approved by a regulatory agency of the Federal or a state government or listed in the U.S. Pharmacopeia, published by the United States Pharmacopeial Convention, Inc., Rockville Md., or other generally recognized pharmacopeia for use in animals, and more particularly in humans.

A “pharmaceutical composition” according to the invention may be present in the form of a composition, wherein the different active ingredients and diluents and/or carriers are admixed with each other, or may take the form of a combined preparation, where the active ingredients are present in partially or totally distinct form. An example for such a combination or combined preparation is a kit-of-parts.

The term “roflumilast” as used in this application refers to roflumilast, its salts, the N-oxide of roflumilast, and its salts, and other metabolites unless specified otherwise or unless it is clear in context that reference is to roflumilast itself.

As used herein, the terms “subject” “or patient” most preferably refers to a human being. The terms “subject” or “patient” may include any mammal that may benefit from the compounds described herein.

A “therapeutic amount” or “therapeutically effective amount” is an amount of a therapeutic agent sufficient to achieve the intended purpose. The effective amount of a given therapeutic agent will vary with factors such as the nature of the agent, the route of administration, the size of the subject to receive the therapeutic agent, and the purpose of the administration. The effective amount in each individual case may be determined empirically by a skilled artisan according to established methods in the art.

As used herein, “treat,” “treating,” or “treatment” of a disease or disorder means accomplishing one or more of the following: (a) reducing the severity and/or duration of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

The present invention relates to the administration of ophthalmic pharmaceutical compositions of the phosphodiesterase-4 inhibitor, roflumilast, for use in treating patients with ocular comorbidities of autoimmune disease. The inventors of the subject application have determined that targeting patients with ocular comorbidities of autoimmune disease, including dry eye disease, can allow for continuous use of existing, systemic medications for the treatment of the underlying autoimmune disease while providing direct and concurrent ophthalmic treatment. The methods disclosed herein can offer an improvement to existing autoimmune treatments, which are often insufficient in treating moderate and severe dry eye disease, among other ocular comorbidities of autoimmune disease, even when adequately treating the systemic disease.

Moreover, addressing ocular complications locally, using the compositions described herein, without the need for additional systemic medication, can avoid myriad issues, including complications due to existing autoimmune treatments, including compounded side effects, drug-drug interactions, and contra-indications.

Roflumilast is a compound of the formula (I):

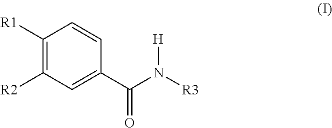

wherein R1 is difluoromethoxy, R2 is cyclopropylmethoxy and R3 is 3,5-dichloropyrid-4-yl.

Roflumilast has the chemical name N-(3,5-dichloropyrid-4-yl)-3-cyclopropylmethoxy-4-difluoromethoxybenzamide. The N-oxide of roflumilast has the chemical name 3-cyclopropylmethoxy-4-difluoromethoxy-N-(3,5-dichloropyrid-4-yl 1-oxide) benzamide. Roflumilast and its synthesis, the use of roflumilast as a phosphodiesterase (PDE) 4 inhibitor, and roflumilast formulations, were described in U.S. Pat. No. 5,712,298, which is incorporated herein by reference. The ophthalmic pharmaceutical composition can include roflumilast as a free base or a pharmaceutically acceptable salt. Exemplary salts of roflumilast are salt described in paragraphs and of U.S. Patent Application Publication No. US 2006/0084684, the disclosure of which is incorporated herein by reference. In certain embodiments, the pharmaceutical composition comprises a metabolite of roflumilast, including the N-oxide of the pyridine residue of roflumilast or salts thereof, as an active ingredient.

In certain embodiments, a method of treating patients with ocular comorbidities of autoimmune disorder is provided. In certain embodiments, the ocular comorbidity of autoimmunity is selected from the group consisting of diseases of both the ocular surface/anterior compartment as well as the posterior compartment of the eye, including primary or secondary dry eye disease, keratitis, corneal ulcers, conjunctivitis, blepharitis, scleritis, episcleritis, uveitis (pan, posterior, anterior, intermediate, or uveitis associated with a systemic disease such as Juvenile Idiopathic Arthritis related uveitis), neurotrophic keratitis, corneal wound healing, corneal melt, glaucoma, cataracts, retinal vasculitis, optic neuritis, macular edema, retinal vein occlusion, choroiditis, retinal detachment, vitreous/retinal hemorrhaging, optic atrophy, photophobia, diplopia, thyroid eye disease, diabetic retinopathy, diabetic macular edema, geographic atrophy, or ocular pain (idiopathic, neuropathic, nociceptive, nociplastic, neuropathic-nociceptive, inflammatory, mixed-mechanism, psychogenic, or multifactorial in origin). In preferred embodiments, the ocular comorbidity is primary dry eye disease or dry eye disease secondary to an additional ophthalmic condition, neurotrophic keratitis, uveitis, diabetic retinopathy, or thyroid eye disease. In especially preferred embodiments, the ocular comorbidity of autoimmune disease is primary or secondary dry eye disease. These ocular comorbidities can be more severe and more resistant to treatment in patients with an autoimmune disorder. See, e.g., Xiong 2024, Henrich 2014. Thus, there is an even greater need to treat these ocular disorders in patients with an autoimmune disorder. In preferred embodiments, the ocular comorbidity is dry eye disease.

In certain embodiments, the method involves selecting a patient that has previously been diagnosed with an autoimmune disorder. In some embodiments, the autoimmune disorder is selected from the group consisting of Graves' disease, Hashimoto's thyroiditis, hypothyroidism, hyperthyroidism, or other thyroid dysfunction, mixed connective tissue disease, scleroderma, CREST syndrome (also known as limited cutaneous systemic sclerosis), rheumatoid arthritis, polyarthritis, sero-negative arthritis, anti-cardiolipin antibody positivity, rheumatoid pleurisy, polymyalgia rheumatica, Sjögren's disease, spondylarthrite, ankylosing spondylitis, multiple sclerosis, lupus (SLE or LN), cutaneous lupus erythematosus, alopecia areata, vitiligo, atopic dermatitis, eczema, psoriasis, psoriatic arthritis, lichen sclerosis, lichen planus, ulcerative colitis, Crohn's disease, diverticulitis, primary biliary cholangitis, type 1 diabetes, LADA, coeliac disease, sarcoidosis, chronic osteoarthritis, primary biliary cholangitis, rosacea, or uveitis (including non-infectious HLA B27 uveitis). In certain embodiments, the patient has one or more autoimmune disorders, a condition known as polyautoimmunity. The autoimmune disorder can be an autoimmune disorder associated with Th1 and/or Th17 driven pathologies. Many autoimmune diseases include pathology associated with activation of Th1 and/or Th17 cells (Markovics 2022, Talaat 2015, Waite 2012, Yasuda 2019, Marwaha 2012), including elevated level of cytokines that activate these cells, and elevated levels of cytokines secreted by activated Th1 and/or Th17 cells. For example, dry eye disease patients with Sjögren's have higher levels of IL-17A (secreted by activated Th 17 cells), IL-6 (activates Th17 cells), and IL-12p80 (activates Th1 cells), compared to non-Sjögren's (Liu 2017; Zhao 2018). Patients with Sjögren's and thyroid disease demonstrate higher tear levels of IL-17, tumor necrosis factor alpha, and IL-6 compared with controls (Lee 2013). In Hashimoto's thyroiditis (HT) patients, dysregulation extends to elevated matrix-metalloproteinase-9 (p<0.001) and IL-6 (p<0.010) compared with non-HT DED and healthy controls (Randelovic 2025). Roflumilast can downregulate cytokine activity, including Th1 and Th17 associated cytokines, as set forth in U.S. Patent Publication No. 2023/0090417A1, which is incorporated herein by reference. Thus, the methods disclosed herein can be used to treat these ocular comorbidities of autoimmune disorders for which there is a high unmet need to treat.

In some embodiments, the patient is selected on the basis of a characteristic, biomarker, and/or signature associated with an autoimmune condition. A biomarker may be indicative of disease etiology, disease-related cytokine burden, activity, progression, or responsiveness or lack of responsiveness to a particular therapeutic regimen, and by selecting patients on the presence of a biomarker may ensure that the treatment is administered to individuals more likely to benefit from therapeutic intervention. In certain embodiments, the patient may be selected based on a serum biomarker. In some embodiments, the patient can be previously diagnosed with any of the autoimmune disorders recited above.

In certain embodiments, the patient population treated using the methods disclosed herein is a patient population with a specific Th1/Th17 therapeutic profile. Without being bound by theory, it is believed that that cytokine or chemokine activity, particularly Th17, Th1, and/or Th2 cytokine activity, driven by inflammatory stress in at least one eye tissue selected from the group consisting of corneal and conjunctival tissue, could be downregulated by administration of roflumilast-containing compounds. Further, in certain embodiments, it is believed that roflumilast may more strongly downregulate cytokine activity associated with both Th1 and Th17 cells, or the combination of Th1 and Th2 and Th17 cells, relative to cytokine downregulation across mixtures of these cell types by other immunosuppressant, immunomodulatory, or non-steroidal anti-inflammatory agents, and more robustly in the conjunctiva than by steroids, cyclosporines, or antihistamines. Given that the conjunctiva is the source of immune cell infiltration (Pflugfelder 2013), potency in the conjunctival region is of particular interest. Lissamine green conjunctival staining scores have been shown to correlate significantly with the expression of Th1 and Th17-related cytokines such as IFNg secreted by activated Th1, IL-6 as an activator of Th17, and IL-17 secreted by activated Th17 (Yang 2019). Thus, it is believed that selecting for the Th17 and Th1 biomarkers in patients with an underlying autoimmune disorder can lead to positive efficacy and safety in immune-based dry eye disease and other ocular manifestations of an autoimmune disorder.

Further, the selection of certain autoimmune patient populations is itself a biomarker for Th17 and Th1 pathology, given the prevalent involvement of these cytokines across many autoimmune populations. Use of a selected group of autoimmune diseases as defined by medical history to act as a biomarker in turn reduces heterogeneity of disease etiology and thereby biological heterogeneity of the target patient population.

In some embodiments, the method can include administering a roflumilast pharmaceutical composition to said patient to treat said ocular comorbidity with regularity. In certain embodiments, the administration can be conducted as a regimen, such as at regular intervals. For example, the administration can be conducted once daily, twice daily, thrice daily, four times daily, once per week, twice per week, three times per week, or four times per week, monthly, or as needed (PRN). In a preferred embodiment, said administration can occur twice daily. In certain embodiments, the administration can be part of a maintenance dose or titrating dose regimen.

In some embodiments, the administration can occur for a prescribed period of time or used chronically. For example, the administration can occur for a period of about two days to at least about six weeks, or until an improvement in the eye condition or disease is observed. Exemplary periods of time for the treatment regimen include one week, two weeks, one month, six weeks, two months, three months, four months, five months, six months, seven months, eight months, nine months, or one year. In a preferred embodiment, the treatment regimen can be tested for eight weeks to represent chronic treatment, or treatment that continues so long as the ocular comorbidities occur. In another embodiment, and as an example, the administration may occur once per week, once per month, once every 2, 3, 4, 5, 6, 7, 8, 9, 10, 11, 12, 13, 14, 15, 16 weeks, once per quarter, once every sixth months, as needed (PRN), per physician direction, or per some clinical criteria such as treat and extend or other criteria, such as chronic use. In some embodiments, the pharmaceutical composition can be administered as an ongoing treatment, with no end.

In certain embodiments, the treatment of the ocular comorbidity can be evaluated by one or more quantitative methods of assessing ocular surface staining or damage in patients with said ocular comorbidity (Wolffsohn, 2017). It is believed that using a corneal- and conjunctival-based staining endpoint can demonstrate the impact of the drug on both the cornea and the conjunctiva, the latter of which is a much more immune-rich target for intervention. In some embodiments, ocular surface staining or damage can be assessed to evaluate patient outcome.

In some embodiments, ocular surface staining or damage can be assessed using lissamine green conjunctival staining (LGCS) according to several relevant staining scales. Total LGCS (tLCGS) is a particularly relevant endpoint for measuring outcomes in dry eye patients with a comorbid autoimmune condition, as the conjunctiva is an immune-rich tissue with relevance to the Th17/Th1 biology of both diseases. In some embodiments, the LGCS score can be graded according to the National Eye Institute (NEI) 0-3 grading scale in a total of 6 regions. In some embodiments, the tLGCS score can be assessed according to the National Eye Institute (NEI) 0-3 grading scale for 6 regions, where the total score is calculated from the 4 non-superior regions (maximum score of 12). In some embodiments, a baseline score can be assessed at a time point prior to commencement of treatment, for example, 0 days, 1 day, 5 days, 7 days, 14, 21 days, or 30 days prior to commencement of treatment. In a preferred embodiment, a baseline score can be assessed prior to (e.g., immediately before) commencement of treatment. In another preferred embodiment, a baseline score can be assessed 0 to 14 days prior to commencement of treatment. In some embodiments, ocular surface staining or damage can be assessed as change in score relative to a baseline. In some embodiments, treatment can be evaluated using tLGCS score, and treatment of said ocular comorbidity may be achieved when a patient achieves a reduction of tLGCS score relative to baseline following treatment. In some embodiments, the score can be assessed at one or more specific time points, for example, before and/or after a given reference point, such as the commencement of treatment. In some embodiments, the score can be assessed after a certain number of days or weeks of commencing treatment, for example, after 1 day, after 1 week, after 2 weeks, after 4 weeks, after 8 weeks, after 12 weeks, or 1, 2, 3, 4, 5, 6, 7, 8, 9, 10, 11, 12 months after commencing treatment. In a preferred embodiment, the score can be assessed after 4, 5, 6, 7, 8, 9, 10, 11, or 12 weeks of commencing treatment.

In certain embodiments, the methods disclosed herein can result in a reduction of tLGCS score following administration relative to baseline. In certain embodiments, the methods can result in a reduction of tLGCS score of 1, 2, 3, or 4 or higher relative to baseline after 4 or 8 weeks of commencing treatment.

In some embodiments, ocular surface staining or damage can be assessed using corneal fluorescein staining (CFS) according to several relevant staining scales. In some embodiments, the CFS score can be graded according to the Lexitas modified NEI 0-4 grading scale covering 5 regions, with a maximum score of 20, or other NEI or Oxford scale of regional staining in the cornea. In some embodiments, a baseline score can be assessed at a time point prior to commencement of treatment, for example, 0 days, 1 day, 5 days, 7 days, 14 days, 21 days, or 30 days prior to commencement of treatment. In a preferred embodiment, a baseline score can be assessed prior to (e.g., immediately before) commencement of treatment. In another preferred embodiment, a baseline score can be assessed 0 to 14 days prior to commencement of treatment. In some embodiments, ocular surface staining or damage is assessed as a change in score relative to a baseline. In some embodiments, treatment can be evaluated using total corneal fluorescein (tCFS) score, and treatment of said ocular comorbidity may be achieved when a patient achieves a reduction of tCFS score relative to baseline following treatment. In some embodiments, the score can be assessed at specific time points, for example, before and after a given reference point, such as the commencement of treatment. In some embodiments, the score can be assessed at one or more specific time points, for example, before and/or after a given reference point, such as the commencement of treatment. In some embodiments, the score can be assessed after a certain number of days or weeks of commencing treatment, for example, after 4 weeks, after 8 weeks, after 12 weeks, or 1, 2, 3, 4, 5, 6, 7, 8, 9, 10, 11, 12 months after commencing treatment. In a preferred embodiment, the score can be assessed after 4-12 weeks of commencing treatment.

In certain embodiments, the methods disclosed herein can result in a reduction of tCFS score following administration relative to baseline. In certain embodiments, the methods can result in a reduction of tCFS score of 1, 2, 3, 4, 5, 6, 7, 8, 9, or 10 or higher relative to baseline after 4 or 8 weeks of commencing treatment.

In certain embodiments, the methods disclosed herein can result in a reduction of central corneal fluorescein staining (cCFS) score following administration relative to baseline. In certain embodiments, the methods can result in a reduction of cCFS score of 0.5, 1, 1.5, or 2 or higher relative to baseline after 4 or 8 weeks of commencing treatment.

In certain embodiments, the methods disclosed herein can result in an improvement in one or more of the following symptomatic assessments of dry eye severity: (a) ocular dryness VAS score; (b) ocular discomfort VAS score; or (c) Ocular Surface Disease Index (OSDI)-dry eye (DE) quality of life outcomes; or other metrics of dry eye related quality of life or specific symptoms such as ocular itch VAS score, etc. (Wolffsohn, 2017, FDA Dry Eye Guidance 2020). In certain embodiments, the methods disclosed herein can result in an improvement of additional exploratory endpoints including ocular itch VAS score; ocular pain VAS score, hyperemia score; regional corneal fluorescein staining; Schirmer's; or tear-film break up time. In certain embodiments, the methods disclosed herein can result in an improvement of Ocular Surface Disease Index (OSDI) score of quality of life following administration relative to baseline.

In certain embodiments, the methods disclosed herein can result in a reduction in ocular pain. In certain embodiments, the ocular pain may be any form of ocular or periocular pain, regardless of origin, severity, or chronicity. In certain embodiments, ocular pain is chronic ocular surface pain (COSP). In other embodiments, the ocular surface pain is acute. In certain embodiments, the ocular pain may be classified as neuropathic, nociceptive, nociplastic, inflammatory, mixed, or combinations thereof. In certain embodiments, the pain can be neuropathic corneal pain. In certain embodiments, the ocular pain can be inflammatory and/or result from a systemic inflammatory condition. In certain embodiments, the ocular pain is associated with autoimmune or inflammatory disease such as Sjogren's syndrome, ocular graft-versus host disease, or celiac disease. In certain embodiments, the ocular pain may be associated with infectious, metabolic, degenerative, or traumatic conditions. In certain embodiments, the ocular pain is associated with eye surgery, extended contact lens wear, shingles, diabetes, trigeminal neuralgia, radiation therapy, dry eye disease, thyroid disease, or thyroid eye disease. In certain embodiments, features of the ocular pain are manifested as light sensitivity, dryness, itch, dysesthesia, burning, prickling, or a crawling feeling on the eye's surface that result in a painful or uncomfortable sensation. In some embodiments, the ocular pain may manifest with photophobia, tearing, foreign body sensation, or combinations of sensory disturbances. In certain embodiments, the degree of pain can be determined by techniques such as the Visual Analog Scale (VAS) ocular pain test, the Ocular Surface Disease Index (OSDI), the Ocular Pain Assessment Survey (OPAS) or the Standardized Patient Evaluation of Eye Dryness (SPEED) technique. In other embodiments, pain assessment may include patient-reported outcomes, physician-administered questionnaires, or imaging-based or neurophysiological biomarkers of pain.

In certain embodiments, the administration results in a reduction of at least 8, 10, 12, 15, 20, 25, 30, 35, or 40 or higher ocular pain VAS score relative to baseline. In certain embodiments, the patient has a baseline ocular pain VAS score of at least 30 signifying moderate to severe levels of pain. In certain embodiments, the patient has a baseline ocular pain VAS score of at least 35. In certain embodiments, the reduction in ocular pain VAS score is obtained after administration of roflumilast for at least eight weeks. In certain embodiments, the reduction in ocular pain VAS score is obtained after administration once daily, twice daily, three times daily, or four times daily.

### Ophthalmic Pharmaceutical Compositions

The ophthalmic pharmaceutical composition administered as part of the methods described herein comprises roflumilast. In certain embodiments, the pharmaceutical composition comprises roflumilast in a range from about 0.01% w/v to about 5.0% w/v, or from about 0.01% w/v to about 3.0% w/v, or from about 0.01% w/v to about 2.0% w/v, or from about 0.01% to about 1.0% w/v, or from about 0.01% to about 0.3% w/v. For example, the pharmaceutical composition may comprise any of the following w/v percents of roflumilast: 0.01%, 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 7%, 1.8%, 1.9%, 1.0%, 2.1%, 2.2%, 2.3%, 2.4%, 2.5%, 2.6%, 2.7%, 2.8%, 2.9%, 3.0%, etc. In a preferred embodiment, the pharmaceutical composition comprises 0.1% w/v of roflumilast. In another preferred embodiment, the pharmaceutical composition comprises 0.3% w/v of roflumilast.

In certain embodiments, the ophthalmic pharmaceutical composition administered as part of the method can be a suspension, solution, eye drops, eye ointments, gels, creams, a spray, nasal spray, an injectable formulation (intravitreal, subconjunctival, sub-tenon, suprachoroidal or other injections), or an adsorbent implant, depot or adsorbent contact lens. In preferred embodiments, the pharmaceutical composition is a suspension, wherein the active ingredient (i.e., roflumilast) is suspended in a pharmaceutical carrier and/or excipients. In certain embodiments, the ophthalmic pharmaceutical composition of roflumilast comprises a viscosity agent, a surfactant, and a buffer. In certain embodiments, the ophthalmic pharmaceutical composition can include one or more additional excipients, including for example, a stabilizer, a preservative, a wetting agent, a diluting agent, a pH adjuster, a tonicity agent, or an absorption enhancer. In certain embodiments, the ophthalmic pharmaceutical composition can also be utilized for anterior or posterior ophthalmic situations in the form of an injection (intravitreal, suprachoroidal, or other), as a depot, an implantable adsorbent device for any ophthalmic or surrounding tissue placement, an in situ forming gel, or a drug/device combination, wherein the active ingredient (i.e., roflumilast) is suspended with one or more of the excipients above, for example a viscosity agent, a surfactant, or a buffer; with or without a device or inert depot compound.

In certain embodiments, the ophthalmic pharmaceutical composition administered as part of the method includes a viscosity agent. In some embodiments, the viscosity agent is at least one selected from the group consisting of hydroxypropyl methylcellulose (HPMC), hydroxyethyl cellulose (HEC), polyvinyl pyrrolidone or povidone (PVP), carboxymethyl cellulose, hypromellose, methylcellulose, or polyvinyl alcohol (PVA). In preferred embodiments, the viscosity agent is PVP. In certain embodiments, the viscosity agent is a dextran or gelatin. In addition, the viscosity agent can include a carbomer in certain embodiments, such as a carbomer copolymer Type A or a carbomer copolymer Type B including those marketed under the trade name Carbopol® by Lubrizol®. In certain embodiments, the ophthalmic pharmaceutical formulation can comprise a viscosity agent in a range from about 0.1% w/v to about 5.0% w/v, or from about 0.1% w/v to about 4.0% w/v, or from about 0.1% w/v to about 3.0% w/v, or from about 0.1% w/v to about 2.0% w/v, or from about 0.1% to about 1.0% w/v, or from about 0.1% to about 0.5% w/v. For example, the ophthalmic pharmaceutical comprises any of the following w/v percents of a viscosity agent: 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 7%, 1.8%, 1.9%, 1.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, etc.

In certain embodiments, the ophthalmic pharmaceutical composition administered as part of the method includes a surfactant. In certain embodiments, the surfactant is at least one selected from the group consisting of polysorbates (including, polysorbate 20, polysorbate 40, polysorbate 60, and polysorbate 80) and tyloxapol. In certain embodiments, the ophthalmic pharmaceutical formulation can comprise a surfactant in a range from about 0.05% w/v to about 3.0% w/v, or from about 0.05% w/v to about 2.0% w/v, or from about 0.05% to about 1.0% w/v, or from about 0.1% to about 0.5% w/v. For example, the ophthalmic pharmaceutical comprises any of the following w/v percents of a surfactant: 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 7%, 1.8%, 1.9%, 1.0%, 2.1%, 2.2%, 2.3%, 2.4%, 2.5%, 2.6%, 2.7%, 2.8%, 2.9%, 3.0%, etc.

In certain embodiments, the ophthalmic pharmaceutical composition administered as part of the method includes a buffer. In certain embodiments, the buffer is at least one selected from the group consisting of citrate, phosphate, Tris-HCl (Tris), acetate, and borate buffers. In certain embodiments, the ophthalmic pharmaceutical formulation can comprise a buffer in a range from about 0.05% w/v to about 7.5% w/v, or from about 0.05% w/v to about 5.0% w/v, or from about 0.05% to about 3.0% w/v, or from about 0.05% w/v to about 2.0% w/v, or from about 0.05% to about 1.0% w/v. For example, the ophthalmic pharmaceutical comprises any of the following w/v percents of a buffer: 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 7%, 1.8%, 1.9%, 1.0%, 2.1%, 2.2%, 2.3%, 2.4%, 2.5%, 2.6%, 2.7%, 2.8%, 2.9%, 3.0%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, etc.

The inventors of the subject application have identified that roflumilast undergoes hydrolysis at low potencies in certain ophthalmic pharmaceutical compositions and under certain standard sterile manufacturing processes. The ophthalmic pharmaceutical compositions can reduce the rate of hydrolysis. In certain embodiments, the pH of the ophthalmic pharmaceutical compositions is between about 6.0 and about 6.7 to reduce the rate of hydrolysis of roflumilast. In preferred embodiments, the pH of the ophthalmic pharmaceutical composition is between about 6.2 and about 6.7, and more preferably between about 6.3 to about 6.6. In preferred embodiments, the osmolality of the ophthalmic pharmaceutical composition is about 270 mOsm/kg to 330 mOsm/kg, more preferably about 270 mOsm/kg to about 300 mOsm/kg, and even more preferably 270 mOsm/kg to 280 mOsm/kg.

In certain embodiments, the ophthalmic pharmaceutical formulation can comprise one or more salts as tonicity agents in a range from about 0.05% w/v to about 5.0% w/v, or from about 0.05% w/v to about 3.0% w/v, or from about 0.05% w/v to about 2.0% w/v, or from about 0.05% w/v to about 1.0% w/v. Suitable salts that may function as tonicity agents include, for example, sodium chloride (NaCl), potassium chloride (KCl), calcium chloride (CaCl₂)), magnesium chloride (MgCl₂), sodium bicarbonate (NaHCO₃), sodium sulfate (Na₂SO₄), sodium citrate, and sodium acetate. In some embodiments, the tonicity agent may include sodium chloride at about 0.3% to about 0.7% w/v, or potassium chloride at about 0.05% w/v. For example, the ophthalmic pharmaceutical formulation can comprise any of the following w/v percents of a salt tonicity agent: 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, etc.

In certain embodiments, the ophthalmic pharmaceutical formulation can comprise one or more polyols as non-ionic tonicity agents in a range from about 0.05% w/v to about 5.0% w/v, or from about 0.05% w/v to about 3.0% w/v, or from about 0.05% w/v to about 2.0% w/v, or from about 0.05% w/v to about 1.0% w/v. Suitable polyols that may function as tonicity agents include, for example, glycerin, mannitol, sorbitol, xylitol, and erythritol. In some embodiments, the tonicity agent may include glycerin at about 2.0% w/v. For example, the ophthalmic pharmaceutical formulation can comprise any of the following w/v percents of a polyol tonicity agent: 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, etc.

In certain embodiments, the ophthalmic pharmaceutical composition does not include any preservatives or anti-microbial agents, as most ophthalmic preservatives and anti-microbial agents are known to cause patient discomfort, burning, stinging, or irritation particularly in the eyes of patients with dry eye disease.

The ophthalmic pharmaceutical compositions administered as part of the methods described herein are stable and exhibit a particle size distribution suitable for ophthalmic delivery. Particle size of the ophthalmic pharmaceutical composition for suspensions can be assessed using laser diffraction methods. Laser diffraction is recognized by standards and guidance agencies including ISO and ASTM and is widely used to determine particle size distributions. In conducting the assessment, the sample is passed through a laser beam, which results in laser light scattered at a range of angles. Detectors placed at fixed angles measure the intensity of light scattered at that position. A mathematical model is then applied to generate a particle size distribution.

In particle size determinations, the median value is defined as the value where half of the population resides above this point, and half resides below this point. For particle size distributions the median is called the D50. The D50 is the size that splits the distribution with half above and half below this diameter. The distribution width may also be characterized by citing one, two or three values on the x-axis, typically some combination of the D10, D50, and D90. The D50 (or the median), as discussed above, refers to the diameter wherein half of the population lies below this value. Similarly, 90 percent of the distribution lies below the D90, and 10 percent of the population lies below the D10.

In certain embodiments, the ophthalmic pharmaceutical composition administered as part of the method exhibits a particle size distribution characterized by a d90 value of less than or equal to about 50 μm prior to preferential processing. In certain embodiments, the ophthalmic pharmaceutical composition exhibits a particle size distribution characterized by a d90 value of from about 5 μm to about 25 μm. In certain embodiments, the pharmaceutical compositions exhibit a particle size distribution characterized by a d90 value of from about 5 μm to about 15 μm. In preferred embodiments, the pharmaceutical compositions exhibit a particle size distribution characterized by a d90 value of less than or equal to 10 μm.

In certain embodiments, the pharmaceutical composition administered as part of the method has a particle size distribution characterized by a d90 value of from about 5 μm to about 25 μm, or more preferably, less than or equal to about 10 μm.

Roflumilast in combination with multiple viscosity agents undergoes particle size growth and aggregation in certain heat transferring ophthalmic pharmaceutical manufacturing processes designed to sterilize a formulation. Avoiding sterilization of roflumilast in the same vessel and at the same time as the inactive ingredients, including excipients and surfactants, can reduce the extent of particle size growth and aggregation while maintaining product potency. Additionally, mixing both sterile API with sterile inactives reduces particle aggregation by reducing the need for additional energy inputs like autoclaving, which can cause particle aggregation. In certain embodiments, slow dry heat sterilization at a temperature less than the melting point of roflumilast, gamma or x-ray radiation, or other methods of sterilization of API can be used to sterilize the roflumilast while standard autoclaving can be used to sterilize the inactives before creating the final mixed formulation for an ophthalmic pharmaceutical composition which is optimized in potency, purity, and particle size, ideal for use in the eye.

In certain embodiments, the sterility and safety of the composition to be administered can be ensured via terminal sterilization followed by sterility testing. With the pharmaceutical compositions listed herein, dry heat or gamma or x-ray radiation can provide assurance of sterility, followed by sterility testing. The accuracy and extent of the gamma radiation is validated via dosimeter recordings of total radiation experienced in all quadrants of the gamma chamber. Further, sterility can be tested via standard two-week screening post sterilization. Both non-clinical and clinical batches have been assessed in this manner, both validating adequate terminal sterilization as well as clearing two-week sterility testing with no microbial growth. In certain embodiments, when using the product/suspension as an injection, the injectable product can be controlled for endotoxins. In certain embodiments, the final injectable product can have less than 1 Endotoxin Units per milliliter (i.e., <1 EU/ml).

In certain embodiments, the active pharmaceutical ingredient (API) can be sterilized by dry heat or by gamma irradiation prior to formulation with inactive ingredients. In certain embodiments, the complete suspension may undergo terminal sterilization in its final container, such as a single-use topical container such as a dropper bottle or blow-fill-seal (BFS) unit dose, a vial or pre-filled syringe, thereby ensuring sterility of both the product and its packaging. In certain embodiments, dry heat sterilization or terminal sterilization can be used for a pharmaceutical composition by topical application, because the composition will be directly applied or injected onto or into the ocular globe or surrounding tissues or chambers (injections may include sub-conjunctivally, intravitreally, suprachoroidally, peribulbarly, retrobulbarly, or injection to another site). Dry heat sterilization and/or terminal sterilization ensures that both the product and the vial are sterile. Needles and syringes which are pre-sterilized are readily available to be used with such a pharmaceutical composition and product configuration. In certain embodiments, a product for injection could also be provided in a pre-filled syringe. In certain embodiments, the resuspendable sterile suspension is provided in a crimp-capped vial, which would be used with separate, pre-sterilized needles to ensure full-process sterility. The pharmaceutical composition can be resuspended by vortexing, shaking, or mixing to resuspend the suspension, and then the product can be drawn through the pre-sterilized needle prior to injection. In certain embodiments, the pre-sterilized needle for drawing suspension can be a lower gauge needle which is then replaced with a higher gauge needle for injection (injection typically accomplished with a 27- to −30-gauge needle) or the same needle could be used for drawing and injection so long as sterile conditions are maintained.

In certain embodiments, the pharmaceutical composition administered as part of the method is terminally sterilized by gamma sterilization and characterized by a retained potency of greater than 99% of the original value of active substances.

The following examples illustrate certain embodiments of the invention without limitation.

### EXAMPLES

While various embodiments have been described herein, it should be understood that they have been presented by way of example only, and not limitation. Thus, the breadth and scope of the present disclosure should not be limited by any of the described exemplary embodiments. Moreover, any combination of the above-described elements in all possible variations thereof is encompassed by the disclosure unless otherwise indicated herein or otherwise clearly contradicted by context.

Four ophthalmic pharmaceutical compositions comprising roflumilast were prepared as set forth in Examples 1-4.

### Example 1

The ophthalmic pharmaceutical composition comprising roflumilast set forth in Table 1 was prepared.

TABLE 1
Ophthalmic Pharmaceutical Suspension of Roflumilast

| | |
|---|---|
| Ingredient | % w/v |

| | | |
|---|---|---|
| Roflumilast | 0.1%, 0.3%, or 1.0% | w/v |
| Hydroxypropyl methylcellulose | 0.3% | w/v |
| Polysorbate 80 | 0.1% | w/v |
| Phosphate/Citrate Buffer | 0.45%/0.05% | w/v |

| | |
|---|---|
| Water for injection | q.s. ad 1.0 mL |

### Example 2

The ophthalmic pharmaceutical compositions comprising roflumilast set forth in Table 2 was prepared.

TABLE 2
Ophthalmic Pharmaceutical Suspension of Roflumilast

| Ingredient | % w/v | |
|---|---|---|
| Roflumilast | 0.1% or 0.3% | w/v |
| Hydroxyethyl cellulose | 0.35% | w/v |
| Polysorbate 80 | 0.1% | w/v |

| | |
|---|---|
| Phosphate/Citrate Buffer | 0.45%/0.05% |
| Water for injection | q.s. ad 1.0 mL |

### Example 3

The ophthalmic pharmaceutical compositions comprising roflumilast set forth in Table 3 were prepared.

TABLE 3
Ophthalmic Pharmaceutical Suspension of Roflumilast

| Ingredient | % w/v | |
|---|---|---|
| Roflumilast | 0.1%, 0.3%, or 1.0% | w/v |
| Polyvinylpyrrolidone | 0.6% | w/v |
| Tyloxapol | 0.3% | w/v |

| | |
|---|---|
| Phosphate/Citrate Buffer | 0.45%/0.05% or 0.25%/0.05% |
| Water for injection | q.s. ad 1.0 mL |

### Example 4

The ophthalmic pharmaceutical composition comprising roflumilast set forth in Table 4 was prepared.

TABLE 4
Ophthalmic Pharmaceutical Suspension of Roflumilast

| Ingredient | % w/v | |
|---|---|---|
| Roflumilast | 0.1% or 0.3% | w/v |
| Carboxymethyl cellulose | 0.5% | w/v |
| Polysorbate 80 | 0.1% | w/v |

| | |
|---|---|
| Phosphate/Citrate Buffer | 0.45%/0.05% |
| Water for injection | q.s. ad 1.0 mL |

### Example 5

The ophthalmic pharmaceutical composition comprising roflumilast set forth in Table 5 was prepared. The formulation in Table 5 is suitable for injection, including intravitreal injection.

TABLE 5
Ophthalmic Pharmaceutical Composition of Roflumilast

| Ingredient | % w/v |
|---|---|
| Roflumilast | 2.0%-5.0% |
| Sodium carboxymethyl cellulose | 0.50% |
| Sodium chloride | 0.60% |
| Polysorbate 20 | 0.15% |
| Potassium chloride | 0.05% |
| Calcium chloride (dihydrate) | 0.05% |
| Magnesium chloride (hexahydrate) | 0.05% |
| Sodium acetate (trihydrate) | 0.10% |
| Sodium citrate (dihydrate) | 0.10% |
| 1N HCl | Adjust to PH ~6.5 |
| Water for injection | q.s. ad 10.0 mL |

### Example 6

An ocular five-day pharmacokinetic preclinical study was conducted in Healthy Dutch Belted Rabbits with the Example 3 formulation for topical application (n=21). The subjects were administered BID dosing of 40 μL of the ophthalmic suspension for four days and QD on the fifth day. Plasma concentrations of roflumilast were measured at certain intervals following the terminal dose. Additionally, the concentration of roflumilast in the cornea and conjunctiva was measured at various time points following the terminal dose.

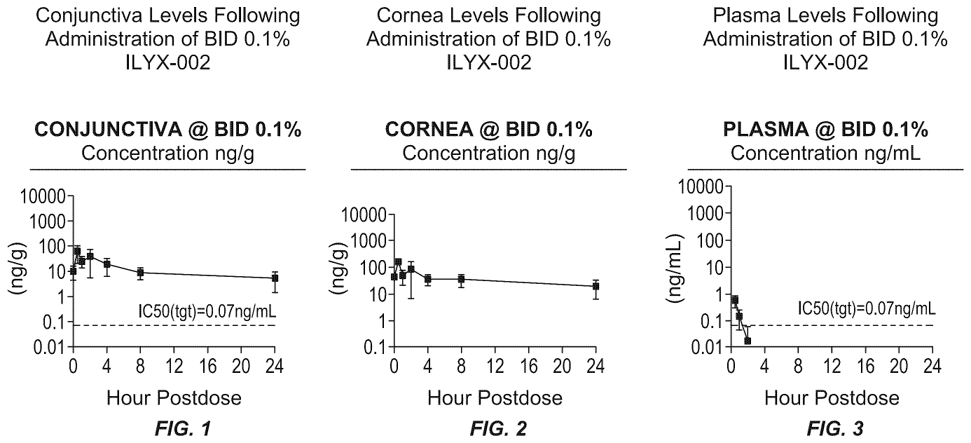

The results of the study in Dutch Belted Rabbits are set forth in FIGS. 1-3. FIG. 1 shows the concentration of roflumilast in the conjunctiva 0, 0.5, 1, 2, 4, 8, and 24 hours after the terminal dose for each group. FIG. 2 shows the concentration of roflumilast in the cornea 0, 0.5, 1, 2, 4, 8, and 24 hours after the terminal dose for each group. FIG. 3 shows the concentration of roflumilast in the plasma after BID dosing for four days and QD dosing on the fifth day. FIG. 3 shows the concentration of roflumilast in the plasma 0, 0.5, 1, 2, 4, 8, and 24 hours after the terminal dose for each group. In FIGS. 1-3, the x-axis is time in hours, and the y-axis is concentration (ng/mL or ng/g) of roflumilast. FIGS. 1 and 2 show that the formulation produced elevated and therapeutic levels of roflumilast in the cornea and conjunctiva following administration. Again, surprisingly, the results indicate that the drug is reaching the ocular surface and anterior areas of the eye, including the cornea and conjunctiva in therapeutic levels, without a corresponding level in the plasma.

FIG. 1 is a graph depicting the concentration of roflumilast in the conjunctiva after five days dosing of a 40 μL dose of 0.1% w/v of roflumilast, with twice a day (BID) dosing for four days, followed by one dose on day 5. The x-axis is time in hours, and the y-axis is concentration (ng/g) of roflumilast. Each time point reflects n=6 eyes. The concentration levels in the conjunctiva indicate a steady state drug concentration has been reached with a value greater than 5 ng/g pre-dose at 0 hours holding steady over the course of 24 hours. The larger surface area of the conjunctiva and its heterogeneity can contribute to the depot of suspension particles in the ocular surface.

FIG. 2 is a graph depicting the concentration of roflumilast in the cornea after five days dosing of a 40 μL dose of 0.1% w/v of roflumilast, with twice a day (BID) dosing for four days, followed by one dose on day 5. The x-axis is time in hours, and the y-axis is concentration (ng/g) of roflumilast. Each time point reflects n=6 eyes. The concentration levels in the cornea indicate a steady state drug concentration has been reached with a value well at or above 35 ng/g pre-dose at 0 hours and holding a steady and high value over the course of 24 hours.

FIG. 3 is a graph depicting the concentration of roflumilast in the plasma after five days dosing of a 40 μL dose of 0.1% w/v of roflumilast, with twice a day (BID) dosing for four days, followed by one dose on day 5. The x-axis is time in hours, and the y-axis is concentration (ng/ml) of roflumilast. The concentration levels in the plasma indicate a decreasing drug concentration in plasma after terminal administration, and those hours with data not shown were BLQ, showing a below therapeutic concentration of roflumilast in the plasma.

### Example 7

Two unique animal studies, one of which is discussed in Example 6, were conducted to assess the concentration of roflumilast in various tissues following administration of different concentrations of Example 3. The concentration in various tissues was assessed following five days of administration of the following dosing regimens: (a) 1% w/v roflumilast three times a day (TID); (b) 0.3% w/v roflumilast three times a day (TID); or (c) 0.1% w/v roflumilast two times a day (BID) as outlined in example 6. In one study encompassing (a) and (b), n=6 Dutch belted rabbits per dose arm were dosed topically three times per day according to arm on days 1 to 4, and on day 5, a single dose was given at hour 0. In a different study with the same methodologies and practices, n=21 Dutch belted rabbits represented in group (c) were dosed two times per day on days 1 to 4, and on day 5, a single dose was given at hour 0. In both studies, under all relevant industry standards and guidelines, 3 animals per time point were euthanized and tissues were collected for bioanalytics at the marked time points: for (a) and (b), at 0.5 hours and 8 hours post dose. In the original study conducted solely with arm (c), a dose curve was created using time points at 0.5, 2, 4, 8, and 24 hours. To reduce the number of animals in the second experiment, for group (a) and (b), only 0.5 and 8 hours were tested.

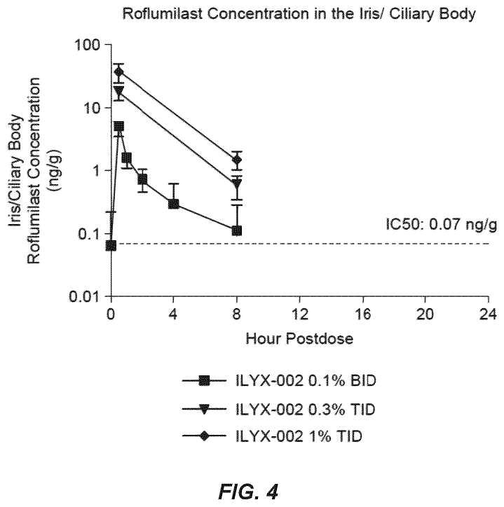

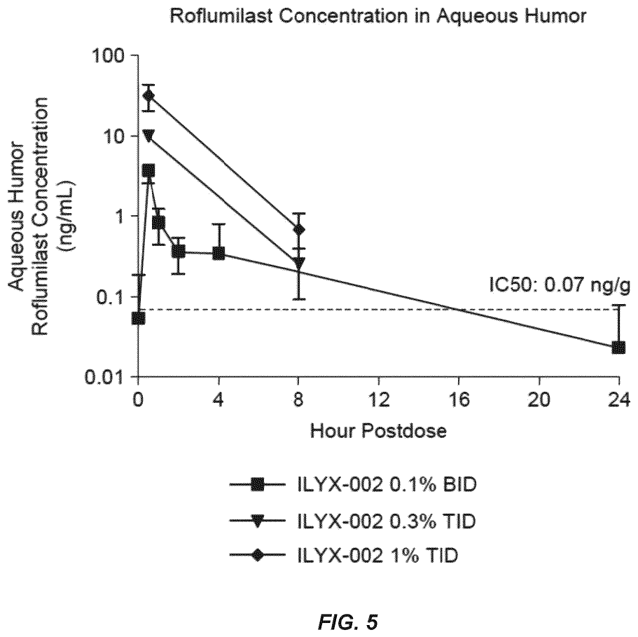

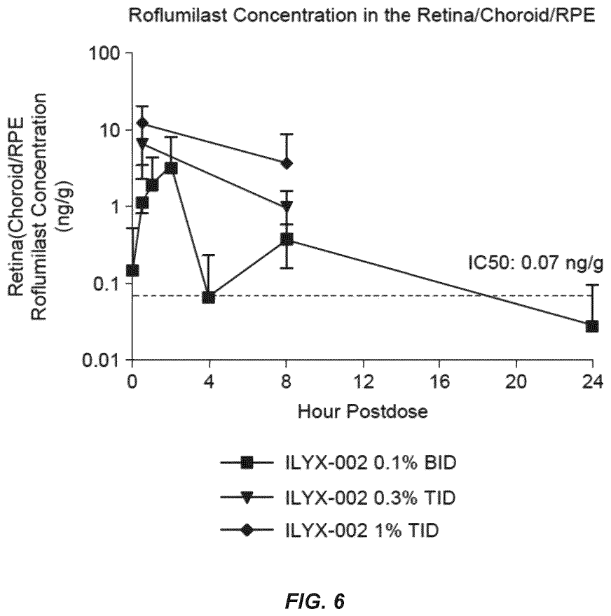

FIG. 4 provides data for the concentration of roflumilast (ng/g) in the iris ciliary body (ICB) as a function of time following: (a) 1% w/v roflumilast administered three times a day; (b) 0.3% w/v roflumilast administered three times a day; or (c) 0.1% w/v roflumilast administered two times a day. FIG. 5 provides data for the concentration of roflumilast (ng/g) in the aqueous humor (AH) as a function of time following: (a) 1% w/v roflumilast administered three times a day; (b) 0.3% w/v roflumilast administered three times a day; or (c) 0.1% w/v roflumilast administered two times a day. FIG. 6 provides data for the concentration of roflumilast (ng/g) in the retina/choroid/retina pigment epithelium (RPE) as a function of time following: (a) 1% w/v roflumilast administered three times a day; (b) 0.3% w/v roflumilast administered three times a day; or (c) 0.1% w/v roflumilast administered two times a day.

Tissue levels of roflumilast were increased with higher doses (1%>0.3%>0.1%) and treatment frequency (three times a day (TID) vs. two times a day (BID)) in the ICB, AH, and retina/choroid/RPE. In the ICB, AH, and retina/choroid/RPE, both 0.3% and 1% provide strong elevation in tissue levels above the IC50 level.

### Example 8

A clinical trial safety run-in was conducted including a cohort of two “sentinel” participants in anticipation of the clinical trial discussed in Example 10. The cohort was conducted at a single-site as a single-masked, non-controlled, staggered-dosing design to evaluate the safety of low-dose (0.1%) roflumilast in Participant 1 and high-dose (0.3%) roflumilast in Participant 2 administered as 1 drop in each eye twice daily (BID). The formulation described in Example 3 was used. The first dose and each subsequent in-clinic dose was administered under direct supervision at the study site and ongoing compliance with administration was assessed by review of bottle returns at each visit. The dosing of participants was staggered, with the safety profile of the initial participant gating the commencement of the second participant.

The patients were previously diagnosed with an autoimmune disorder and were suffering from moderate to severe dry eye disease. The first patient had dry eye disease and was previously diagnosed with Graves' disease. The second sentinel patient had dry eye disease and was previously diagnosed with lupus, scleroderma and Raynaud's disease (an example of poly-autoimmunity). This second patient had significant medical management of their polyautoimmunity with the ongoing use of hydroxychloroquine, prednisone, mycophenolate and denosumab systemically, and yet still had quite severe dry eye disease.

The safety and tolerability was assessed by ocular and non-ocular key safety monitoring assessments elicited by spontaneous reporting by the participant or non-leading inquiry, intraocular pressure (IOP) assessed by tonometry, best corrected visual acuity (BCVA) measured by visual assessment with Snellen chart, external eye examination via slit-lamp biomicroscopy, assessment of the retina via dilated ophthalmoscopy and ocular tolerability as reported by participants in VAS questionnaires. The results on day 1 (D1), day 29 (D29), and day 57 (D57) are set forth in Table 6. OD and OS refer to the right eye and left eye, respectively. OU refers to both eyes. Two sequential IOP measurements were performed and the average of the 2 is presented as IOP (average) in Table 6.

TABLE 6
Safety and Tolerability Results.

| | | Participant #1 | | | Participant #2 | | |
|---|---|---|---|---|---|---|---|
| | | D 1 | D 29 | D 57 | D 1 | D 29 | D 57 |
| BCVA | OD | 20/15 | 20/20 | 20/15 | 20/25 | 20/20 | 20/20 |
| | OS | 20/20 | 20/20 | 20/15 | 20/20 | 20/20 | 20/25 |
| IOP | OD | 18.5 | 18 | 20 | 11 | 13 | 10 |
| (average) | OS | 19 | 19 | 19 | 12.5 | 14 | 12 |
| Ocul. Toler | OU | 15 | 4 | 3 | 68 | 3 | 15 |
| VAS₀₋₁₀₀ | | | | | | | |
| Adverse | 0 adverse events reported | | | | 2 adverse events unrelated | | |
| Events | | | | | | | |

The therapeutic effect of the roflumilast topical ophthalmic suspension of Example 3, administered twice daily (BID) was assessed in terms of the total lissamine green conjunctival staining (tLGCS) score according to the National Eye Institute (NEI) 0-3 grading scale for 6 regions, where the total was calculated from only the 4 non-superior regions (maximum score=12). The therapeutic effect was also assessed in terms of the total corneal fluorescein staining (tCFS) score according to the Lexitas modified NEI 0-4 grading scale for 5 regions, where the total was calculated from all 5 regions (maximum 20). The therapeutic effect was also examined using a Schirmer test, tear-film break up test (TBUT), and hyperemia test. The therapeutic endpoints were assessed at days-7, 1, 29, and 57 post-commencement of eye drops. For the two sentinel patients, certain therapeutic endpoints were also assessed at day 85, with an additional safety follow up visit of symptom endpoints only, 14 days later. The study as set forth in Example 10, however, was later amended to conclude at day 57. Thus, some data presented herein is presented through day 57 to conform with the length of the amended study.

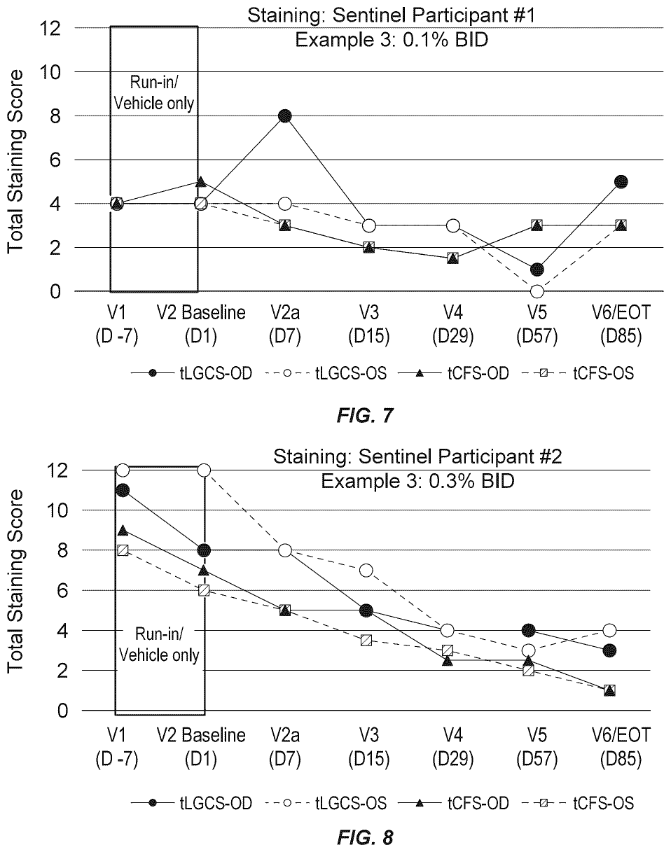

The results through day 57 are set forth in Tables 7 (Participant 1) and 8 (Participant 2). CFB refers to change from baseline, and MCID refers to the estimated minimum clinically important difference estimated based on published data, regulatory approvals, and/or medical opinions. The TBUT and hyperemia values are averages of two measurements. The hyperemia values are nasal and temporal averages on a 4-point scale. OD and OS refer to the right eye and left eye, respectively. The tLGCS and tCFS results for the two sentinel patients are also set forth in FIGS. 7 and 8.

TABLE 7
Therapeutic “Signs” Efficacy Results for Participant 1.

| Efficacy: “Signs” | | D −7 | D 1 | D 29 | D 57 | CFB | MCID |
|---|---|---|---|---|---|---|---|
| Total LGCS | OD | 4 | 4 | 3 | 1 | −3 | −0.25 |
| | OS | 4 | 4 | 3 | 0 | −4 | |
| Total CFS | OD | 4 | 5.0 | 1.5 | 3.0 | −2.0 | −0.25 |
| | OS | 4 | 4.0 | 1.5 | 3.0 | −1.0 | |
| Schirmer (mm) | OD | 17 | 16 | 26 | 14 | −2 | + |
| | OS | 9 | 15 | 19 | 5 | −10 | |
| TBUT (secs, average) | OD | 3.89 | 6.13 | 7.52 | 6.95 | +0.82 | + |
| | OS | 5.48 | 7.28 | 12.0 | 8.49 | +1.21 | |
| Hyperemia | OD | 3.5 | 3.0 | 2.5 | 2.5 | −0.5 | −.25 to −.5 |
| (average: temp + nasal) | OS | 3.5 | 3.5 | 2.5 | 2.0 | −1.5 | |

TABLE 8
Therapeutic “Signs” Efficacy Results for Participant 2.

| Efficacy: “Signs” | | D −7 | D 1 | D 29 | D 57 | CFB | MCID |
|---|---|---|---|---|---|---|---|
| Total LGCS | OD | 11 | 8 | 4 | 4 | −4 | −0.5 |
| | OS | 12 | 12 | 4 | 3 | −9 | |
| Total CFS | OD | 9 | 7.0 | 2.5 | 2.5 | −4.5 | −0.5 |
| | OS | 8 | 6.0 | 3.0 | 2.0 | −4.0 | |
| Schirmer (mm) | OD | 1 | 1 | 3 | 1 | 0 | + |
| | OS | 1 | 1 | 4 | 3 | +2 | |
| TBUT (secs, average) | OD | 5.17 | 6.19 | 6.47 | 7.41 | +1.22 | + |
| | OS | 7.38 | 6.64 | 7.73 | 6.11 | −0.53 | |
| Hyperemia | OD | 2.0 | 2.0 | 1.0 | 1.0 | −1.0 | −.25 to −.5 |
| (average: temp + nasal) | OS | 2.5 | 2.0 | 1.5 | 1.5 | −0.5 | |

The two patients were also assessed using the symptom index (SI) VAS 0-100 scale. Participants were instructed to report how severe, on average, their symptoms were related to dry eye disease over the past 24 hours. A mark of 0% corresponded to “no discomfort” and 100% corresponded to “maximal discomfort.” The results at days-7, 0 (baseline), 7, 15, 29, and 57 are set forth in Tables 9 (Participant 1) and 10 (Participant 2). CFB refers to change from baseline, and MCID refers to the estimated minimum clinically important difference estimated based on published data, regulatory approvals, and/or medical opinions.

TABLE 9
Therapeutic “Symptoms” Efficacy Results for Participant 1.

| Efficacy: “Symptoms”⁴ | | | | | | | |
|---|---|---|---|---|---|---|---|
| VAS 0-100 | | D −7 | D 1 | D 29 | D 57 | CFB | MCID |
| General Ocular Discomfort | OU | 70 | 74 | 40 | 30 | −44 | −5 to −10 |
| Burning/Stinging | OU | 59 | 69 | 20 | 10 | −59 | |
| Itching | OU | 19 | 35 | 20 | 9 | −26 | |
| Foreign Body Sensation | OU | 66 | 17 | 22 | 11 | −6 | |
| Dryness | OU | 70 | 74 | 10 | 14 | −60 | |
| Photophobia | OU | 61 | 53 | 2 | 13 | −40 | |
| Pain | OU | 32 | 42 | 3 | 13 | −29 | |

TABLE 10
Therapeutic “Symptoms” Efficacy Results for Participant 2.

| Efficacy: “Symptoms”⁴ | | | | | | | |
|---|---|---|---|---|---|---|---|
| VAS 0-100 | | D −7 | D 1 | D 29 | D 57 | CFB | MCID |
| General Ocular Discomfort | OU | 72 | 58 | 23 | 21 | −37 | −5 |
| Burning/Stinging | OU | 63 | 57 | 20 | 12 | −45 | to −10 |
| Itching | OU | 60 | 28 | 29 | 19 | −9 | |
| Foreign Body Sensation | OU | 68 | 56 | 21 | 21 | −35 | |
| Dryness | OU | 82 | 60 | 49 | 15 | −45 | |
| Photophobia | OU | 66 | 82 | 39 | 51 | −31 | |
| Pain | OU | 15 | 8 | 19 | 14 | +6 | |

The results suggest that 0.1% and 0.3% (w/v) roflumilast produced a clinically meaningful response in the sentinel patients following 57 days of treatment. In some respects, the administration of roflumilast outperformed existing therapies. For example, Restasis (OTX-101 0.1% BID) produced a reduction in 0.4 and 0.8 tCFS after 4 weeks in the Phase 3 ESSENCE 2 and Phase 2b/3 ESSENCE trials, respectively. Miebo (perfluorohexyloctane) produced a reduction in tCFS of 1 to 1.2 after 8 weeks in the GOBI and MOJAVE phase 3 trials, respectively. Cequa (0.09% cyclosporine) produced a reduction of 0.4 after 12 weeks in a Phase 3 trial. These results suggest that BID administration of roflumilast 0.1% and 0.3% is effective at treating dry eye disease in patients previously diagnosed with autoimmune disorders. The results show this clinically meaningful effect not just in the cornea, the most common site of drug residence for multiple agents, but also in the conjunctiva, which is uniquely targeted with strong therapeutic levels of the roflumilast topical suspension.

### Example 9

A trial was conducted in eleven participants, including one of the participants (Participant 1) from Example 6. The patients were administered 0.1% roflumilast as 1 drop in each eye twice daily. 7 participants were followed to d57 for efficacy. The formulation of Example 3 was used. The patients were previously diagnosed with an autoimmune disorder and were suffering from moderate to severe dry eye disease.

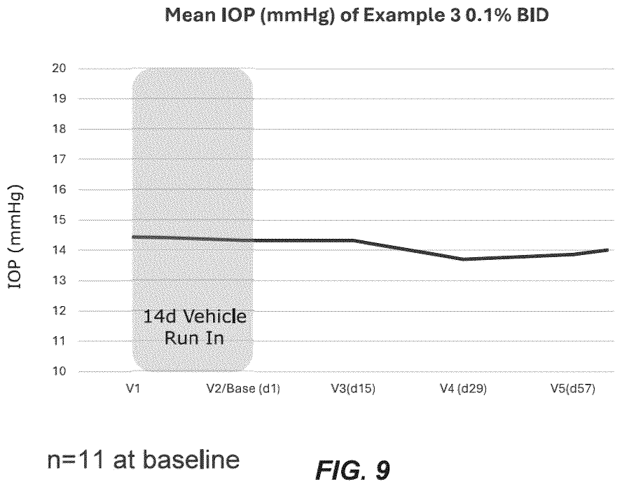

The intraocular pressure (IOP) in patients administered 0.1% w/v roflumilast (Example 3) was measured over time. The results of the study are reported in FIG. 9. The results reflect that patients' intraocular pressure was stable over 3 months following administration of the formulation of Example 3. Because steroids cause a notable rise in intraocular pressure, which can lead to glaucoma, the invention represents a significant safety improvement over use of steroids.

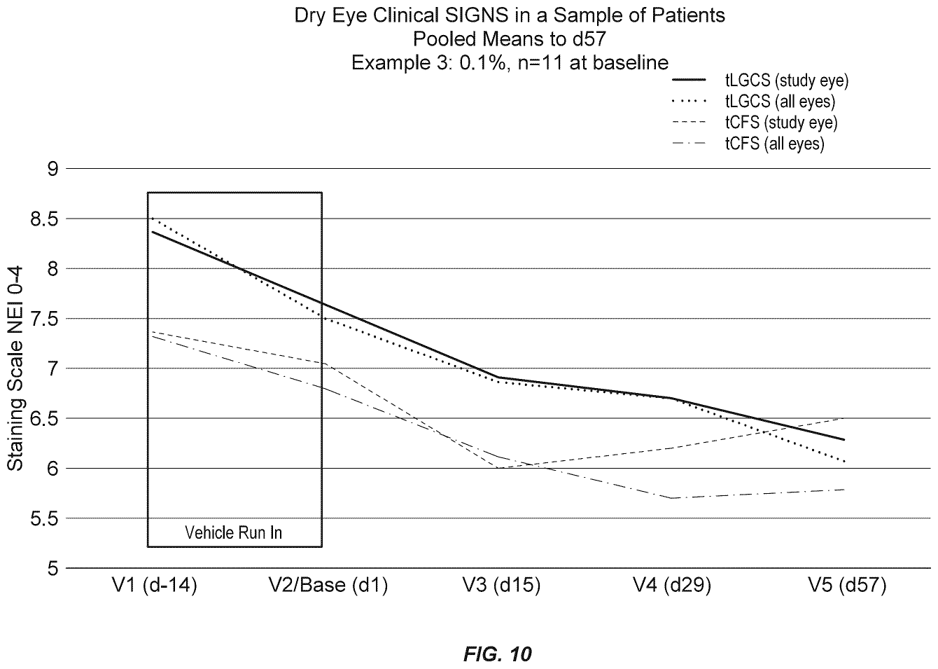

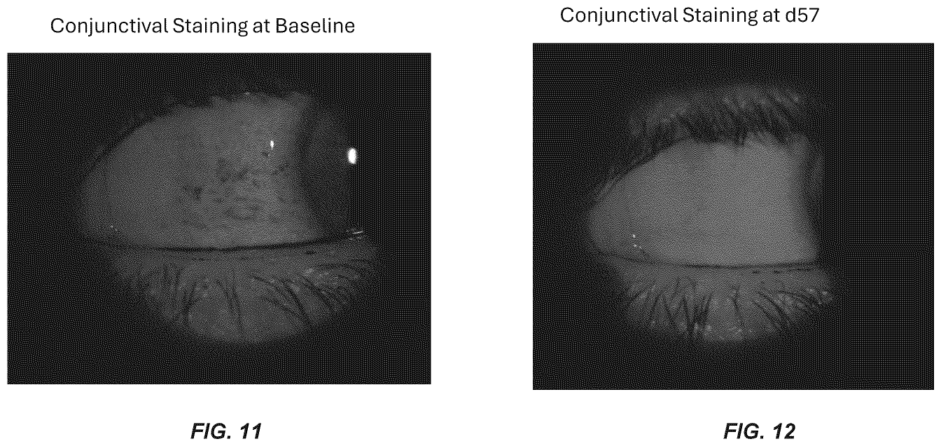

The therapeutic effect on the objective signs of the roflumilast topical ophthalmic suspension of Example 3, administered twice daily was assessed in terms of the total lissamine green conjunctival staining (tLGCS) score according to the National Eye Institute (NEI) 0-3 grading scale for 6 regions, where the total score was calculated from the 4 non-superior regions (maximum score of 12). The therapeutic effect was also assessed in terms of the total corneal fluorescein staining (tCFS) score according to the Lexitas modified NEI 0-4 grading scale for 5 regions, where the total was calculated from all 5 regions (maximum score of 20). The therapeutic effect was also examined using a Schirmer test, tear-film break up test (TBUT), and hyperemia test. The therapeutic endpoints were assessed at days-7, 1, 29, and 57, days post-commencement of eye drops. The results are set forth in FIG. 10. The pooled mean results for each timepoint show that tLGCS and tCFS scores generally went down over the course of the study, which indicates that 0.1% w/v roflumilast was therapeutically effective in treating dry eye disease. Additionally, FIG. 11 shows an exemplary total lissamine green conjunctival staining in a patient at day 1 (baseline). FIG. 12 shows an exemplary total lissamine green conjunctival staining in the same patient after BID administration of 0.1% w/v roflumilast on day 57.

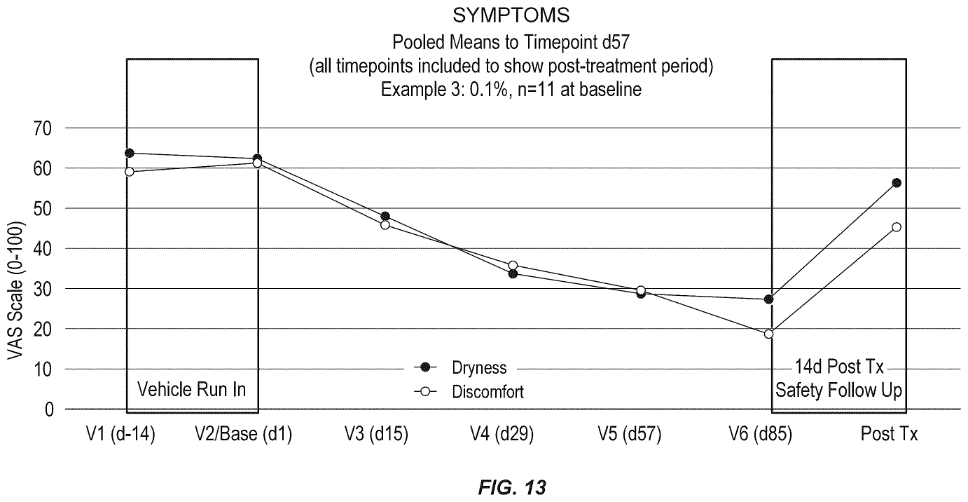

The therapeutic effect was also measured using the following symptomatic endpoints: (a) ocular dryness VAS score; (b) ocular discomfort VAS score; and (c) Ocular Surface Disease Index (OSDI)-dry eye (DE) outcomes. The therapeutic endpoints were assessed, where applicable, at days-14, 1, 29, 57, and out to 85 days post-commencement of eye drops, as well as 14 days post-treatment termination (regardless of whether that treatment was at d28, 57, or 85). The pooled mean results for each timepoint are set forth in FIG. 13. The results show that ocular dryness VAS scores and ocular discomfort VAS scores generally went down over the course of the study, which indicates that BID administration of 0.1% w/v roflumilast was therapeutically effective in treating dry eye disease. Additionally, following the completion of the administration of drug, the symptoms returned in the participants as reflected in the last data point on the right.

### Example 10

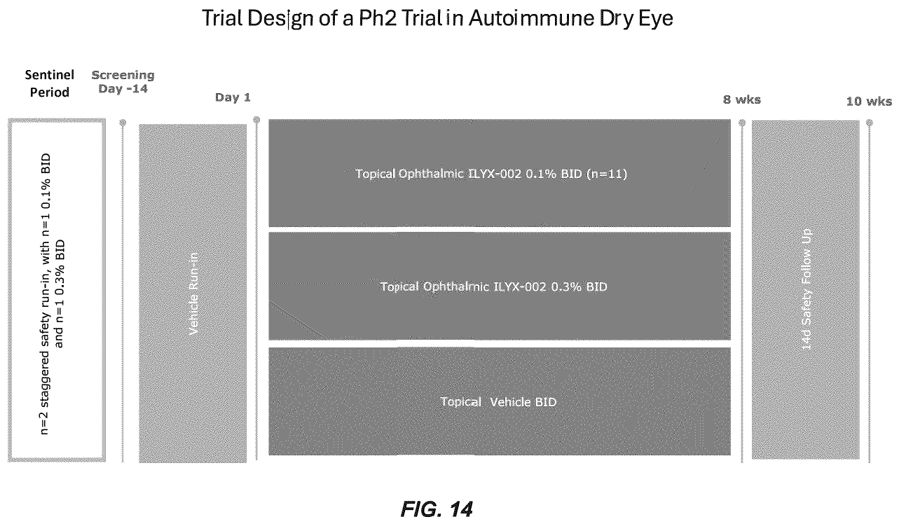

A Phase 2 clinical trial was designed to assess the tolerability, safety, and potential therapeutic effect of repeat dosing of a roflumilast topical ophthalmic suspension. A roflumilast topical ophthalmic suspension was compared to vehicle control in participants with dry eye disease with autoimmune disorder. FIG. 14 provides an illustration of the trial design.

Participants were randomized to one of the following two treatment groups in a 1:1 ratio as follows:

| | |
|---|---|
| Group 1 | Ophthalmic Pharmaceutical Suspension of Roflumilast of |
| | Example 3 at 0.1% (1 drop into each eye twice daily |
| | through to Day 57) |
| Group 2 | Ophthalmic Pharmaceutical Suspension of Roflumilast of |
| | Example 3 at 0.3% (1 drop into each eye twice daily |
| | through to Day 57) |
| Group 3 | Vehicle (1 drop into each eye twice daily through to |
| | Day 57) |

Group 1 of the study consisted of the 11 patients discussed in Example 9, who received the low dose (0.1%) administered BID, from day 1 up to 85 days. Two patients in Group 1 completed 85 days of the study. The study was later amended to extend to 57 days and enrollment of Group 1 was stopped. Group 2 of the study serves as the active arm at a higher dose (0.3%) administered BID. Group 3 serves as the control arm, administered BID. Participants in group 3 received vehicle, which is the same composition as a roflumilast topical ophthalmic suspension but without the active ingredient.

The first dose and each subsequent in-clinic dose were administered under direct supervision at the study site and the participant continued twice-daily dosing at home until the next visit. Ongoing compliance with administration was assessed by review of bottle returns at each visit.

Participants were selected on the basis of several inclusion criteria. Participants were male or females between and inclusive of the ages of 18 and 85. Participants were previously diagnosed with one or more of the following autoimmune disorders: Graves' disease, Hashimoto's thyroiditis, hypothyroidism, hyperthyroidism, or other thyroid dysfunction, mixed connective tissue disease, scleroderma, CREST syndrome (also known as limited cutaneous systemic sclerosis), rheumatoid arthritis, polyarthritis, sero-negative arthritis, anti-cardiolipin antibody positivity, rheumatoid pleurisy, polymyalgia rheumatica, Sjögren's disease, spondylarthrites, ankylosing spondylitis, multiple sclerosis, lupus (SLE or LN), cutaneous lupus erythematosus, alopecia areata, vitiligo, atopic dermatitis, eczema, psoriasis, psoriatic arthritis, lichen sclerosis, lichen planus, ulcerative colitis, Crohn's disease, diverticulitis, primary biliary cholangitis, type 1 diabetes, latent autoimmune diabetes in adults (LADA), coeliac disease, sarcoidosis, chronic osteoarthritis, primary biliary cholangitis, rosacea, or uveitis (including non-infectious HLA B27 uveitis). Some patients had one or more autoimmune disorders, a condition known as polyautoimmunity. Participants had a best-corrected visual acuity (BCVA) of 20/100 or better in trial eye. Participants had moderate to severe dry eye disease. Participants used artificial tears at least 2 times per day for at least 30 days prior to screening.

Participants were excluded on the basis of several key exclusion criteria. Included participants did not have a known hypersensitivity or contraindications to the trial treatment or its components. Participants did not, within 30 days prior to screening, take contraindicated medications or investigational treatments. Participants did not use contact lenses during the trial. Participants did not have a history of ocular surface or anterior segment surgery within 12 months of screening. Participants did not have a history of uncontrolled glaucoma or actively being treated for glaucoma. Participants did not have a history of punctal cautery. Participants did not have a current use of punctal plugs. Participants did not have hepatic insufficiency. Participants were not currently pregnant or lactating. Importantly for participants who may be reliant on certain systemic medications for the treatment of their systemic autoimmune disease, participants were allowed, but were not going to change, in dose or frequency, within 90 days prior to screening, or anticipated during trial, specified chronic medications for their autoimmune or other conditions.

Safety and tolerability were assessed by ocular and non-ocular key safety monitoring assessments elicited by spontaneous reporting by the participant or non-leading inquiry, intraocular pressure assessed by tonometry, best corrected visual acuity measured by visual assessment with Snellen chart, external eye examination via slit-lamp biomicroscopy, assessment of the retina via dilated ophthalmoscopy and ocular tolerability as reported by participants in questionnaires. After Day 57, participants continued to be monitored for safety for 14 days until Day 71.

The therapeutic effect of the roflumilast topical ophthalmic suspension of Example 3, administered twice daily, as compared to vehicle, was assessed in terms of the total lissamine green conjunctival staining (tLGCS) score. The therapeutic endpoint was the change from baseline to Week 8 in the tLGCS score, in the trial eye, graded according to the National Eye Institute (NEI) 0-3 grading scale (6 regions: Temporal, Superior Temporal, Inferior Temporal, Superior Nasal, and Inferior Nasal). The tLGCS score was the sum of the 4 non-superior region scores (Temporal, Inferior Temporal, Nasal, and Inferior Nasal), with a total range from 0 to 12. The therapeutic endpoint was assessed at days-14, 1, 15, 29, 57 post-commencement of eye drops.

The therapeutic effect of the roflumilast topical ophthalmic suspension of Example 3, administered twice daily, as compared to vehicle, was also assessed in terms of the total corneal fluorescein staining (tCFS) score. The therapeutic endpoint was the change from baseline to Week 8 in the tCFS score, in the trial eye, graded according to the Lexitas modified NEI 0-4 grading scale. The therapeutic endpoint was assessed at days-14, 1, 15, 29, 57 post-commencement of eye drops.

The therapeutic effect was also measured using the following exploratory endpoints: (a) ocular dryness VAS score; (b) ocular discomfort VAS score; (c) Ocular Surface Disease Index (OSDI)-dry eye (DE) outcomes; and additional exploratory endpoints including (d) ocular itch VAS score; (e) ocular pain VAS score, (f) hyperemia score; (g) regional corneal fluorescein staining; (h) Schirmer's; or (i) tear-film break up time.

The first dose and each subsequent in-clinic dose was administered under direct supervision at the study site and ongoing compliance with administration is being assessed by review of bottle returns at each visit. The dosing of sentinel participants was staggered, with the safety profile of the initial sentinel participant gating the commencement of the second sentinel participant. Safety evaluation of each treatment arm at a minimum of 7 days of dosing gated initiation of the Phase 2 trial.

Adverse event data was collected until day 71 (or day 99 for the sentinel patients and certain Group 1 patients) post-commencement of eye drops, or 14 days after termination of drug if a patient stopped treatment early. Slit-lamp biomicroscopy/external eye examination, best corrected visual acuity and tonometry assessments was completed on days-14, 1, 15, 29, 57 and 71 post-commencement of eye drops. Ocular tolerability assessments was completed on days-14, 1, 15, 29, 57 and 71 post-commencement of eye drops. Dilated ophthalmoscopy was completed on days-14, 1 and 57 post-commencement of eye drops.

The results of the study indicated that the drug product was considered safe and well tolerated, with early signs of efficacy sustained through end of treatment. Treatment-emergent adverse events were generally mild to moderate in severity. Discontinuations due to adverse events were infrequent and occurred similarly across treatment groups. Mean VAS OT scores indicated treatment was well tolerated. IOP remained stable, with a mean change of −0.21 mmHg from baseline at Week 8.

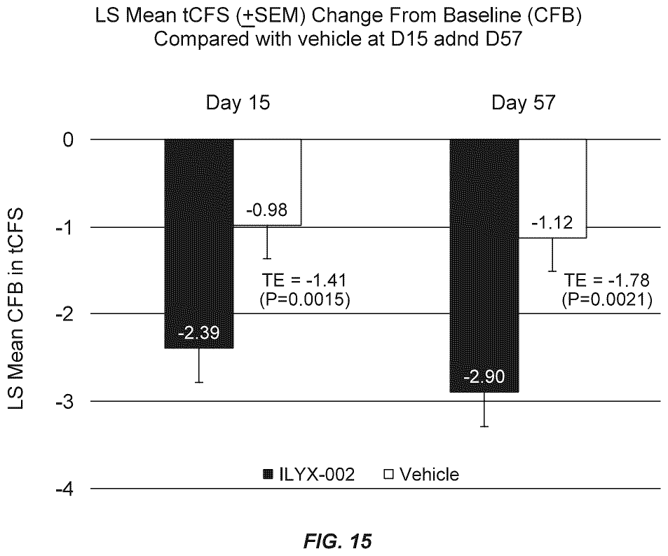

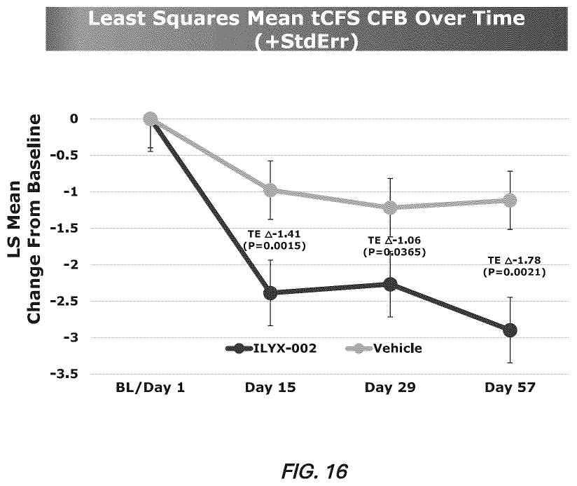

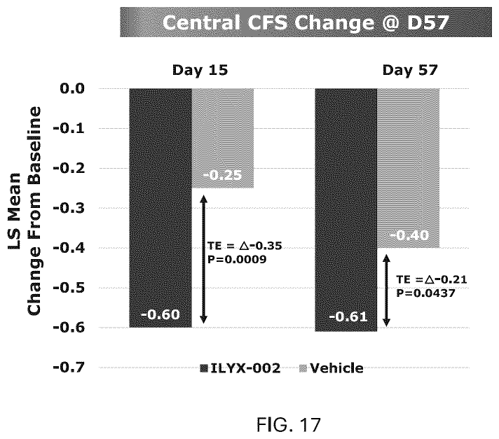

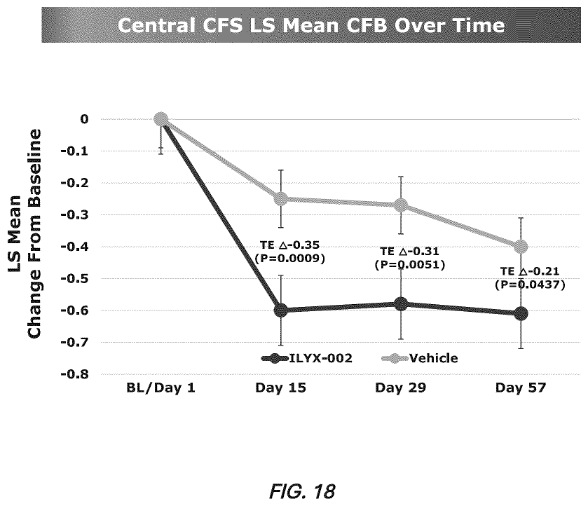

The mean tCFS score (±SD) in the roflumilast treatment group decreased from 6.61±1.99 at baseline to 3.61±2.68 at day 57 (Week 8), with a difference of −2.90±3.03 and a statistically significant mean change of −1.78 (P=0.0021) compared to vehicle as shown in FIGS. 15 and 16. The values were graded according to the Lexitas modified NEI 0-4 grading sale (5 regions), where the tCFS is the sum of all five regions, the total range is 0-20, and the maximum change is less than or equal to 10 based on inclusion parameters. The onset of action was quick, with a mean change by Day 15 of −2.41±2.15 in the treatment arm and a statistically significant difference of −1.41 (P=0.0015) compared to vehicle as shown in FIGS. 15 and 16. The cCFS score in the treatment arm decreased from 0.98±0.118 at baseline to 0.36±0.074 at day 57 (Week 8) with a statistically significant difference of −0.21 (P=0.0437) compared to vehicle as shown in FIGS. 17 and 18. Again, the onset of action was quick, with a statistically significant mean change from baseline observed at day 15 of −0.35 (P=0.0009) compared to vehicle as shown in FIGS. 17 and 18.

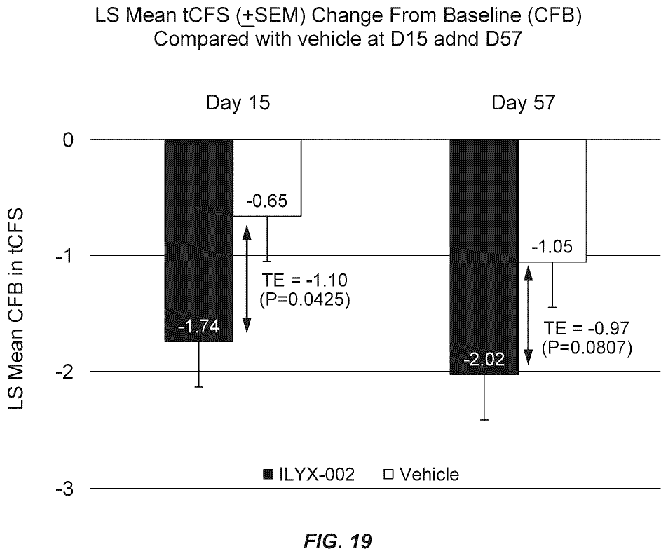

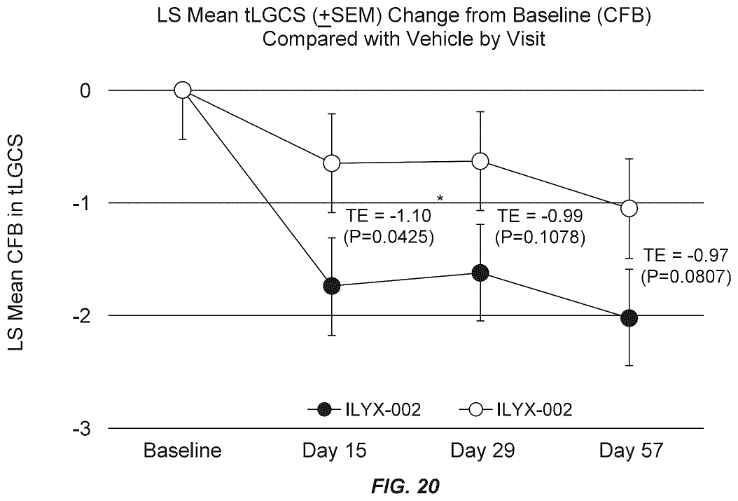

The mean (±SD) baseline total Lissamine Green Conjunctival Staining (tLGCS) scores were 6.9±2.5 for the ILYX-002 treatment arm and 7.6±2.6 for the vehicle arm, with a difference in starting baseline scores of 0.7. The onset of action of roflumilast was observed by Day 15 with a least squared (LS) means change from baseline of −1.74 (95% CI: −2.48 to −1.01) and a treatment effect of −1.10 (95% CI: −2.14 to −0.05; p=0.0425) when compared with vehicle as shown in FIGS. 19 and 20. The magnitude of effect was consistent throughout the treatment period. At Day 57, the least squares (LS) mean change of roflumilast and vehicle were-2.02 (95% CI: −2.79 to −1.25) and −1.05 (95% CI: −1.82 to −0.28), respectively, and the overall treatment effect of roflumilast compared with vehicle was −0.97 (95% CI: −2.05 to 0.11; p=0.0807) as shown in FIGS. 19 and 20.

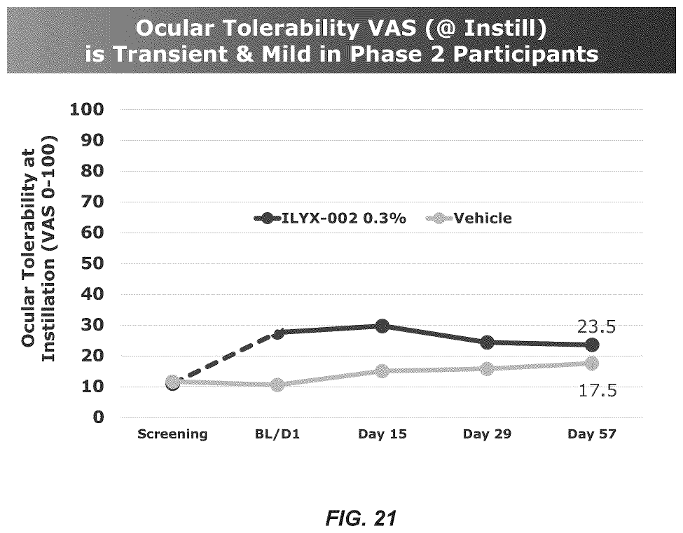

The visual analog scale (VAS) was used for measuring severity of various symptoms of dry eye disease. Participants were asked to rate the severity of each of the following ocular symptoms over the past 24 hours (prior to in-office treatment). A mark of 0% corresponds to “no discomfort” and 100% correspond to “maximal discomfort.” Symptoms assessed included eye discomfort, burning/stinging in eye, itching (pruritus) in eye, foreign body sensation in eye, eye dryness, photophobia, and eye pain. There was a notable change observed from baseline after 57 days for all symptoms in both roflumilast and vehicle treatment arms as shown in FIG. 21.

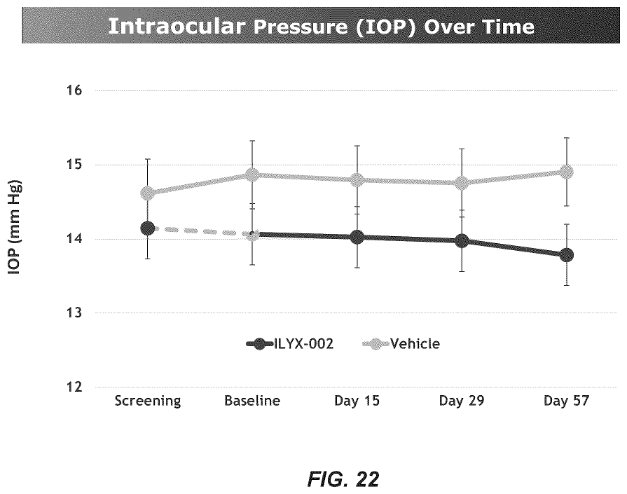

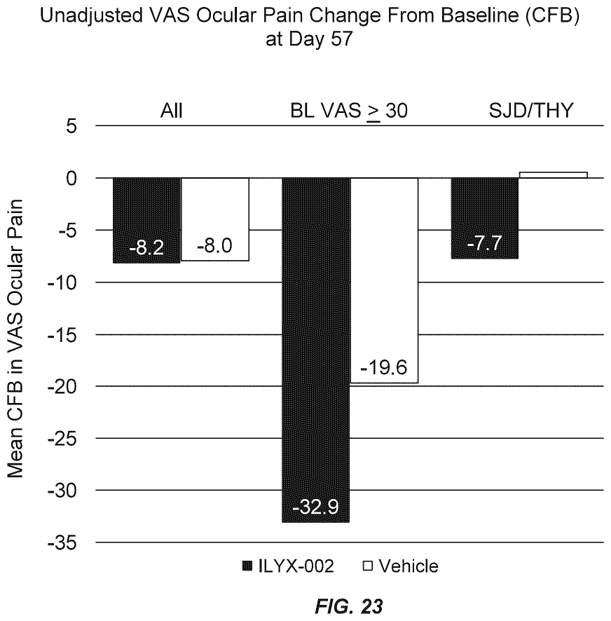

Intraocular pressure (IOP) was monitored at the dates of screening and at baseline followed by days 15, 29, and 57. Observed IOP values and change from baseline values were similar in both the roflumilast and vehicle arms as shown in FIG. 22. At day 57, the mean change from baseline for participants in the roflumilast and vehicle arms was −0.21±2.15 and −0.13±2.04, respectively.

Visual Analog Scale (VAS) ocular pain was monitored at baseline/Day 1 followed by days 15, 29, and 57. VAS ocular pain is measured by having a patient mark a point on a line reflecting the intensity of pain they are experiencing at the reported time point, with one end of the line representing “no pain” while the opposite end represents the “worst pain.” The distance from the “zero” end of the line to the mark is measured and recorded. Table 11 reports the mean, median, and standard deviation of VAS ocular pain scores of all participants in the study that reported data at Baseline and Day 57 who were treated with either ILYX-002 (n=49) or vehicle (n=48), as well as the change from baseline (CFB), treatment effect (Tx Effect), and percent change from baseline (% CFB). Large baseline imbalances were noted between the treatment groups in the overall participant population. To normalize the baseline imbalances, an ANCOVA analysis in which baseline is a covariate was performed and the least squared (LS) means CFB with standard error (Std Err) and treatment effect data is presented in Table 12. Table 13 reports similar data as Table 11 from those participants who were experiencing moderate to severe baseline (BL) VAS ocular pain (i.e. a baseline VAS Pain score of greater than or equal to 30 out of 100) at the start of the study. Table 14 reports data from an ANCOVA analysis, using baseline as a covariate, on the subpopulation of participants who were experiencing moderate to severe baseline (BL) VAS ocular pain (i.e. a baseline VAS Pain score of greater than or equal to 30 out of 100) at the start of the study and includes least squared (LS) means CFB with standard error (Std Err), treatment effect and P-value. Table 15 reports, similar to Table 11 and Table 13, data for the subpopulation of participants who had Sjogren's disease (SJD) or thyroid disease (THY) before the start of the study.

TABLE 11
VAS Ocular Pain in the Overall Population

| All Participants (VAS Pain) | |
|---|---|
| ILYX-002 (n = 49) | Vehicle (n = 48) |

| | Baseline | Day 57 | Baseline | Day 57 |
|---|---|---|---|---|
| Mean | 26.4 | 18.2 | 33.6 | 25.6 |
| Median | 20 | 10 | 26 | 17.5 |
| Std Dev | 22.5 | 20.9 | 27.3 | 27.1 |
| CFB | | −8.2 | | −8 |
| TX Effect | | −0.2 | | |
| % CFB | | −31% | | −24% |

TABLE 12
VAS Ocular Pain ANCOVA Analysis in Overall Population

| | All Participants (VAS Pain) at Day 57 | |
|---|---|---|
| | ILYX-002 (n = 52) | Vehicle (n = 50) |
| LS Mean CFB (Std Err) | −10.0 (3.1) | −5.8 (3.2) |
| Treatment Effect (Std | −4.2 (4.5) | — |
| Err) | | |

TABLE 13
VAS Ocular Pain in Participants with BL ≥ 30

| BL ≥ 30 (VAS Pain) | |
|---|---|
| ILYX-002 (n = 17) | Vehicle (n = 23) |

| | Baseline | Day 57 | Baseline | Day 57 |
|---|---|---|---|---|
| Mean | 53.6 | 20.7 | 57.7 | 38.1 |
| Median | 59 | 10 | 57 | 30 |
| Std Dev | 11.9 | 23.0 | 19.5 | 29.0 |
| CFB | | −32.9 | | −19.6 |
| Tx Effect | | −13.3 | | |
| % CFB | | −61% | | −34% |

TABLE 14
VAS Ocular Pain ANCOVA Analysis
in Participants with BL ≥ 30

| | BL ≥ 30 (VAS Pain) at Day 57 | |
|---|---|---|
| | ILYX-002 (n = 17) | Vehicle (n = 23) |
| LS Mean CFB (Std Err) | −33.8 (6.1) | −19.0 (5.3) |
| Treatment Effect | −14.8 (8.1) | |
| (Std Err) | | |
| P-Value | 0.0746 | |

TABLE 15
VAS Ocular Pain in Participants with Sjogren's
or Thyroid Disease with any BL score

| Sjogren's/Thyroid (VAS Pain) | |
|---|---|
| ILYX-002 (n = 20) | Vehicle (n = 19) |

| | Baseline | Day 57 | Baseline | Day 57 |
|---|---|---|---|---|
| Mean | 25.6 | 17.9 | 27.5 | 28.1 |
| Median | 19.5 | 9 | 15 | 19 |
| Std Dev | 22.3 | 21.7 | 24.5 | 29.2 |
| CFB | | −7.65 | | 0.53 |
| Tx Effect | | −8.2 | | |
| % CFB | | −30% | | +2% |

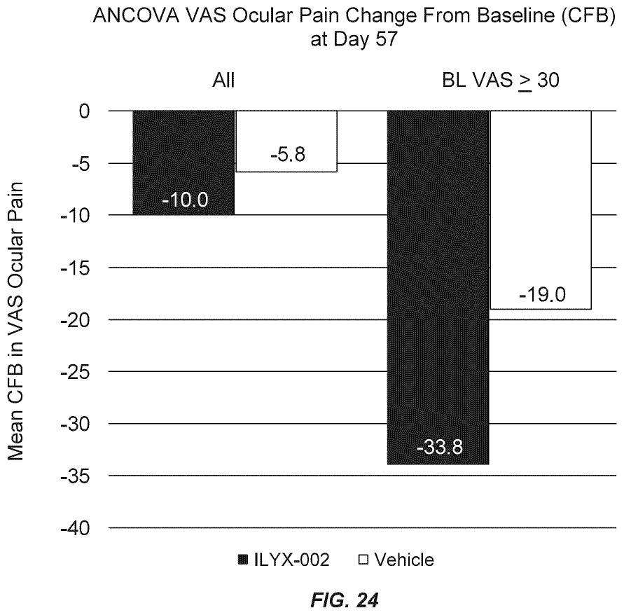

FIG. 23 is a bar graph depicting the unadjusted change from baseline (CFB) at Day 57 in VAS ocular pain among (a) all participants (“All Pts”), (b) those participants with baseline VAS ocular pain greater than or equal to 30 at baseline (“VAS Pain≥30 @BL”), and (c) those with Sjogren's or thyroid disease (SJD/THY). Among all participants, the unadjusted difference in change from baseline between the ILYX-002 and vehicle-treated groups appeared relatively small (−8.2 vs. −8.0) but this has to be considered in the context of the starting baseline imbalances among the treatment groups (refer to ANCOVA analysis data presented in Table 12 and FIG. 24). However, in the subset of participants with comparatively high baselines in the moderate to severe range for VAS ocular pain (VAS Pain≥30 @BL), the unadjusted difference in CFB between the ILYX-002-treated and vehicle-treated groups was surprisingly pronounced (−32.9 vs. −19.6) in this sizeable subpopulation (˜50% of the total sample of the trial). In the Sjogren's/Thyroid population, the unadjusted difference in CFB between the ILYX-002-treated and vehicle-treated groups was unexpectedly stark, particularly compared with the total participants (All Pts) group. The ILYX-002-treated group exhibited a CFB of −7.7 while the vehicle-treated group showed no effect on relieving VAS ocular pain. Indeed, even a slight increase in VAS ocular pain was reported, unlike that seen in the drug-treated population.

FIG. 24 is a bar graph depicting an ANCOVA analysis, using baseline as a covariate, reporting least squared (LS) means CFB data at Day 57 in VAS ocular pain among (a) all participants (“All Pts”) and (b) those participants with baseline VAS ocular pain greater than or equal to 30 at baseline (“VAS Pain≥30 @BL”) for the overall population (“All Pts”). When baselines were included as a covariate in the ANCOVA analysis, the large baseline imbalances between treatment groups were accounted for and a treatment effect signal was observed. When applying the ANCOVA analysis to the subset of participants in which baseline pain was reported as greater than or equal to 30 (“VAS Pain≥30@BL”), the large overall improvement in pain in the ILYX-002-treated group was maintained, resulting in a clinically meaningful and significant treatment effect (Std Err) of −14.8 (8.1), P-value=0.0746.

A high level of inflammation is but one driver of pain. Given that autoimmune patients in the study population have a highly activated inflammatory cascade of Th17 and Th1 cytokines from their underlying disease, they can tend to have higher pain at baseline. However, in more severe dry eye disease, patients can actually lose nerve sensation. As such it is surprising that these autoimmune dry eye patients still have a moderate to severe ocular pain component, and that the reduction of their inflammation with a PDE4 inhibitor can also then control their pain. Steroids are typically given for dry eye pain related flares, and yet they have a significant risk of side effects. An alternate to steroids that can control inflammation and as such, pain, would meet a large need in the treatment of ocular pain, and ties well to the findings that the agents also improve quality of life for these patients.

The foregoing description has been presented for purposes of illustration and description. This description is not intended to limit the invention to the precise form disclosed. A person of ordinary skill in the art will appreciate that modifications and substitutions of the basic inventive description may be made.

What is claimed is:

1. A method of treating an ocular comorbidity of an autoimmune disorder in a patient with an autoimmune disorder, comprising:
selecting a patient previously diagnosed with the autoimmune disorder; and
administering an ophthalmic pharmaceutical composition comprising roflumilast to the patient to treat the ocular comorbidity of the autoimmune disorder.

2. The method of claim 1, wherein the ocular comorbidity is selected from the group consisting of diseases of both the ocular surface/anterior compartment as well as the posterior compartment of the eye, including primary or secondary dry eye disease, keratitis, corneal ulcers, conjunctivitis, blepharitis, scleritis, episcleritis, uveitis, neurotrophic keratitis, corneal wound healing, corneal melt, glaucoma, cataracts, retinal vasculitis, optic neuritis, macular edema, retinal vein occlusion, choroiditis, retinal detachment, vitreous/retinal hemorrhaging, optic atrophy, photophobia, diplopia, thyroid eye disease, diabetic retinopathy, diabetic macular edema, geographic atrophy, or ocular pain.

3. The method of claim 2, wherein the ocular comorbidity is primary or secondary dry eye disease.

4. The method of claim 1, wherein the autoimmune disorder is selected from the group consisting of Graves' disease, Hashimoto's thyroiditis, hypothyroidism, hyperthyroidism, or other thyroid dysfunction, mixed connective tissue disease, scleroderma, CREST syndrome, rheumatoid arthritis, polyarthritis, sero-negative arthritis, anti-cardiolipin antibody positivity, rheumatoid pleurisy, polymyalgia rheumatica, Sjögren's disease, spondylarthrites, ankylosing spondylitis, multiple sclerosis, lupus, cutaneous lupus erythematosus, alopecia areata, vitiligo, atopic dermatitis, eczema, psoriasis, psoriatic arthritis, lichen sclerosis, lichen planus, ulcerative colitis, Crohn's disease, diverticulitis, primary biliary cholangitis, type 1 diabetes, latent autoimmune diabetes in adults, coeliac disease, sarcoidosis, chronic osteoarthritis, primary biliary cholangitis, rosacea, or uveitis.

5. The method of claim 1, wherein the patient has one or more autoimmune disorders.

6. The method of claim 1, wherein the ophthalmic pharmaceutical composition is administered to the patient once daily, twice daily, three times a day or four times a day.

7. The method of claim 6, wherein the ophthalmic pharmaceutical composition is administered to the patient for eight weeks or twelve weeks.

8. The method of claim 7, wherein the patient achieves a reduction of at least 1 total lissamine green conjunctival staining score relative to baseline following treatment.

9. The method of claim 8, wherein the patient achieves a reduction of at least 2 total lissamine green conjunctival staining score relative to baseline following treatment.

10. The method of claim 7, wherein the patient achieves a reduction of at least 1 total corneal fluorescein staining score relative to baseline following treatment.

11. The method of claim 10, wherein the patient achieves a reduction of at least 2 total corneal fluorescein staining score relative to baseline following treatment.

12. The method of claim 1, wherein the ophthalmic composition comprises between 0.1% and 0.3% w/v roflumilast.

13. The method of claim 1, wherein the patient maintains or achieves an improvement in ocular and non-ocular key safety monitoring assessments relative to baseline, including at least one selected from the group consisting of: intraocular pressure assessed by tonometry, corrected visual acuity measured by visual assessment with Snellen chart, external eye examination via slit-lamp biomicroscopy, assessment of the retina via dilated ophthalmoscopy, or ocular tolerability following treatment.

14. A method of treating dry eye disease in a patient, said method comprising:
selecting a patient previously diagnosed with an autoimmune disorder and having dry eye disease; and
administering an ophthalmic pharmaceutical composition comprising roflumilast to the patient to treat the dry eye disease.

15. The method of claim 14, wherein the autoimmune disorder is selected from the group consisting of Graves' disease, Hashimoto's thyroiditis, hypothyroidism, hyperthyroidism, or other thyroid dysfunction, mixed connective tissue disease, scleroderma, CREST syndrome, rheumatoid arthritis, polyarthritis, sero-negative arthritis, anti-cardiolipin antibody positivity, rheumatoid pleurisy, polymyalgia rheumatica, Sjögren's disease, spondylarthrites, ankylosing spondylitis, multiple sclerosis, lupus, cutaneous lupus erythematosus, alopecia areata, vitiligo, atopic dermatitis, eczema, psoriasis, psoriatic arthritis, lichen sclerosis, lichen planus, ulcerative colitis, Crohn's disease, diverticulitis, primary biliary cholangitis, type 1 diabetes, latent autoimmune diabetes in adults, coeliac disease, sarcoidosis, chronic osteoarthritis, primary biliary cholangitis, rosacea, or uveitis.

16. The method of claim 14, wherein the patient has one or more autoimmune disorders, a condition known as polyautoimmunity.

17. The method of claim 14, wherein the ophthalmic pharmaceutical composition is administered to the patient once daily, twice daily, three times a day or four times a day.

18. The method of claim 14, wherein the ophthalmic pharmaceutical composition is administered to the patient for eight weeks.

19. The method of claim 14, wherein the patient achieves a reduction of at least 1 total lissamine green conjunctival staining score relative to baseline following treatment.

20. The method of claim 19, wherein the patient achieves a reduction of at least 2 total lissamine green conjunctival staining score relative to baseline following treatment.

21. The method of claim 14, wherein the patient achieves a reduction of at least 1 total corneal fluorescein staining score relative to baseline following treatment.

22. The method of claim 21, wherein the patient achieves a reduction of at least 2 total corneal fluorescein staining score relative to baseline following treatment.

23. The method of claim 14, wherein the ophthalmic composition comprises between 0.1% and 0.3% w/v roflumilast.

24. The method of claim 14, wherein the patient maintains or achieves an improvement in ocular and non-ocular key safety monitoring assessments relative to baseline, including at least one selected from the group consisting of: intraocular pressure assessed by tonometry, corrected visual acuity measured by visual assessment with Snellen chart, external eye examination via slit-lamp biomicroscopy, assessment of the retina via dilated ophthalmoscopy, or ocular tolerability following treatment.

25. A method of treating ocular pain in a patient, said method comprising:
administering an ophthalmic pharmaceutical composition comprising roflumilast to the patient to treat the ocular pain,
wherein the administration results in a reduction of at least 8 ocular pain Visual Analog Score (VAS) score relative to baseline following treatment.

26. The method of claim 25, wherein the patient has a baseline ocular pain VAS score of at least 30 signifying moderate to severe pain.

27. The method of claim 25, wherein the ophthalmic pharmaceutical composition is administered to the patient once daily, twice daily, three times a day or four times a day for eight weeks.

28. The method of claim 25, wherein the ocular pain is chronic ocular surface pain (COSP), acute ocular surface pain, neuropathic corneal pain, neuropathic nociceptive pain, or nociplastic pain.

29. The method of claim 25, wherein the patient has an autoimmune or inflammatory disease.

30. The method of claim 29, wherein the autoimmune or inflammatory disease is selected from Sjogren's syndrome, thyroid disease, ocular graft-versus host disease, or celiac disease.
